SEQUENCE LISTING (28 sequences)

SEQ ID NO: 1 - protein, 58 aa, Artificial Sequence
  affinity ligand
NAAMDDFAQYSAWAEIEALPNLTEMQKDQFIWSLMFDPSVSKEVLGEAQKLNDSQAPK

SEQ ID NO: 2 - protein, 56 aa, Artificial Sequence
  affinity ligand
AMDDFAQYSAWAEIEALPNLTEMQKDQFIWSLMFDPSVSKEVLGEAQKLNDSQAPK

SEQ ID NO: 3 - protein, 58 aa, Artificial Sequence
  affinity ligand
NAAMDDFDQYAAWAEIEALPNLTEMQKDQFIWSLMFDPSVSLEILAEAKKLNDAQAPK

SEQ ID NO: 4 - protein, 56 aa, Artificial Sequence
  affinity ligand
AMDDFDQYAAWAEIEALPNLTEMQKDQFIWSLMFDPSVSLEILAEAKKLNDAQAPK

SEQ ID NO: 5 - protein, 58 aa, Artificial Sequence
  affinity ligand
  VARIANT (8)..(8): Xaa may be selected from A or D
  VARIANT (11)..(11): Xaa may be selected from S or A
  VARIANT (42)..(42): Xaa may be selected from K or L
  VARIANT (44)..(44): Xaa may be selected from V or I
  VARIANT (46)..(46): Xaa may be selected from G or A
  VARIANT (49)..(49): Xaa may be selected from Q or K
  VARIANT (54)..(54): Xaa may be selected from S or A
NAAMDDFXQYXAWAEIEALPNLTEMQKDQFIWSLMFDPSVSXEXLXEAXKLNDXQAPK

SEQ ID NO: 6 - protein, 233 aa, Artificial Sequence
  fusion protein 214410 comprising affinity ligand of SEQ ID NO 1
NAAMDDFAQYSAWAEIEALPNLTEMQKDQFIWSLMFDPSVSKEVLGEAQKLNDSQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKC

SEQ ID NO: 7 - protein, 231 aa, Artificial Sequence
  fusion protein 214411 comprising affinity ligand of SEQ ID NO 2
AMDDFAQYSAWAEIEALPNLTEMQKDQFIWSLMFDPSVSKEVLGEAQKLNDSQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKC

SEQ ID NO: 8 - protein, 233 aa, Artificial Sequence
  fusion protein 214412 comprising affinity ligand of SEQ ID NO 3
NAAMDDFDQYAAWAEIEALPNLTEMQKDQFIWSLMFDPSVSLEILAEAKKLNDAQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKC

SEQ ID NO: 9 - protein, 58 aa, Artificial Sequence
  non Ig binding protein
IAAQHDKIQQAADKEILHLPNLTEEQRNKFRQSLRDDPSVSAEILAEAKKLNDAQAPK

SEQ ID NO: 10 - protein, 76 aa, Artificial Sequence
  ubiquitin mutein 211883
MQIFVKTLTGKTITLAVKPNDTILKVKVWIEIKEGIPPDQQRLIWAGKQLEDGRTLSDYNIQKESTLHLVLRLRAA

SEQ ID NO: 11 - protein, 82 aa, Artificial Sequence
  ubiquitin mutein 139819
MQIFVKTLTYNPMRYGKTITLEVEPSDTIENVKAKIQDKEGIPPDQQRLIWAGKQLEDGRTLSDYNIRNPDILHLVLRLRAA

SEQ ID NO: 12 - protein, 80 aa, Artificial Sequence
  ubiquitin mutein 215208
MQIFVKTLTVYEVGKTITLEVEPSDTIENVKAKIQDKEGIPPDQQRLIWAGKQLEDGRTLSDYNIFALPMLELLLTLRAA

SEQ ID NO: 13 - protein, 82 aa, Artificial Sequence
  ubiquitin mutein 215220
MQIFVKTLTFKIIPAGKTITLEVEPSDTIENVKAKIQDKEGIPPDQQRLIWAGKQLEDGRTLSDYNIDWPAMLTLSLPLRAA

SEQ ID NO: 14 - protein, 155 aa, Artificial Sequence
  ubiquitin mutein 77404
MRIWVHTLTGKTITLEVEPSDTIENVKAKIQDKEGIPPDQQRLIWAGKQLEDGRTLSDYNINPKLSLHLVLRLRAAGIGMQIFVHTQTGKTITLEVEPSDTIENVKAKIQDKEGIPPDQQRLIWAGKQLEDGRTLSDYNIGWQAPLHLVLRLRAA

SEQ ID NO: 15 - protein, 76 aa, Artificial Sequence
  ubiquitin mutein 77404-1
MRIWVHTLTGKTITLEVEPSDTIENVKAKIQDKEGIPPDQQRLIWAGKQLEDGRTLSDYNINPKLSLHLVLRLRAA

SEQ ID NO: 16 - protein, 75 aa, Artificial Sequence
  ubiquitin mutein 77404-2
MQIFVHTQTGKTITLEVEPSDTIENVKAKIQDKEGIPPDQQRLIWAGKQLEDGRTLSDYNIGWQAPLHLVLRLRA

SEQ ID NO: 17 - protein, 152 aa, Artificial Sequence
  ubiquitin mutein 200053
MQIFVRTQTGKTITLEVEPSDTIENVKAKIQDKEGIPPDQQRLIWAGKQLEDGRTLSDYNIGWAHRLHLVLRLRAAMQIFVHTNTGKTITLEVEPSDTIENVKAKIQDKEGIPPDQQRLIWAGKQLEDGRTLSDYNIWTETILHLVLRLRAA

SEQ ID NO: 18 - protein, 76 aa, Artificial Sequence
  ubiquitin mutein 200053-1
MQIFVRTQTGKTITLEVEPSDTIENVKAKIQDKEGIPPDQQRLIWAGKQLEDGRTLSDYNIGWAHRLHLVLRLRAA

SEQ ID NO: 19 - protein, 76 aa, Artificial Sequence
  ubiquitin mutein 200053-2
MQIFVHTNTGKTITLEVEPSDTIENVKAKIQDKEGIPPDQQRLIWAGKQLEDGRTLSDYNIWTETILHLVLRLRAA

SEQ ID NO: 20 - protein, 76 aa, Artificial Sequence
  ubiquitin
MQIFVKTLTGKTITLEVEPSDTIENVKAKIQDKEGIPPDQQRLIFAGKQLEDGRTLSDYNIQKESTLHLVLRLRAA

SEQ ID NO: 21 - protein, 58 aa, Artificial Sequence
  affinity ligand 187029
NAARSDEAQYSAWAEIEALPNLTEMQKDQFIWSLMFDPSVSKEVLGEAQKLNDSQAPK

SEQ ID NO: 22 - protein, 251 aa, Artificial Sequence
  Fusion protein comprising SEQ ID NO 2 and SEQ ID NO 1 as affinity ligands
AMDDFAQYSAWAEIEALPNLTEMQKDQFIWSLMFDPSVSKEVLGEAQKLNDSQAPKQAPKVDAKFDQAPKVDAKFDNAAMDDFAQYSAWAEIEALPNLTEMQKDQFIWSLMFDPSVSKEVLGEAQKLNDSQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKC

SEQ ID NO: 23 - protein, 137 aa, Artificial Sequence
  Dimer of SEQ ID NO 21
NAARSDEAQYSAWAEIEALPNLTEMQKDQFIWSLMFDPSVSKEVLGEAQKLNDSQAPKQAPKVDAKFDQAPKVDAKFDNAARSDEAQYSAWAEIEALPNLTEMQKDQFIWSLMFDPSVSKEVLGEAQKLNDSQAPKC

SEQ ID NO: 24 - protein, 215 aa, Artificial Sequence
  trimer of SEQ ID NO 21
NAARSDEAQYSAWAEIEALPNLTEMQKDQFIWSLMFDPSVSKEVLGEAQKLNDSQAPKQAPKVDAKFDQAPKVDAKFDNAARSDEAQYSAWAEIEALPNLTEMQKDQFIWSLMFDPSVSKEVLGEAQKLNDSQAPKQAPKVDAKFDQAPKVDAKFDNAARSDEAQYSAWAEIEALPNLTEMQKDQFIWSLMFDPSVSKEVLGEAQKLNDSQAPKC

SEQ ID NO: 25 - protein, 293 aa, Artificial Sequence
  tetramer of SEQ ID NO 21
NAARSDEAQYSAWAEIEALPNLTEMQKDQFIWSLMFDPSVSKEVLGEAQKLNDSQAPKQAPKVDAKFDQAPKVDAKFDNAARSDEAQYSAWAEIEALPNLTEMQKDQFIWSLMFDPSVSKEVLGEAQKLNDSQAPKQAPKVDAKFDQAPKVDAKFDNAARSDEAQYSAWAEIEALPNLTEMQKDQFIWSLMFDPSVSKEVLGEAQKLNDSQAPKQAPKVDAKFDQAPKVDAKFDNAARSDEAQYSAWAEIEALPNLTEMQKDQFIWSLMFDPSVSKEVLGEAQKLNDSQAPKC

SEQ ID NO: 26 - protein, 137 aa, Artificial Sequence
  dimer of SEQ ID NO 1
NAAMDDFAQYSAWAEIEALPNLTEMQKDQFIWSLMFDPSVSKEVLGEAQKLNDSQAPKQAPKVDAKFDQAPKVDAKFDNAAMDDFAQYSAWAEIEALPNLTEMQKDQFIWSLMFDPSVSKEVLGEAQKLNDSQAPKC

SEQ ID NO: 27 - protein, 215 aa, Artificial Sequence
  trimer of SEQ ID NO 1
NAAMDDFAQYSAWAEIEALPNLTEMQKDQFIWSLMFDPSVSKEVLGEAQKLNDSQAPKQAPKVDAKFDQAPKVDAKFDNAAMDDFAQYSAWAEIEALPNLTEMQKDQFIWSLMFDPSVSKEVLGEAQKLNDSQAPKQAPKVDAKFDQAPKVDAKFDNAAMDDFAQYSAWAEIEALPNLTEMQKDQFIWSLMFDPSVSKEVLGEAQKLNDSQAPKC

SEQ ID NO: 28 - protein, 293 aa, Artificial Sequence
  tetramer of SEQ ID NO 1
NAAMDDFAQYSAWAEIEALPNLTEMQKDQFIWSLMFDPSVSKEVLGEAQKLNDSQAPKQAPKVDAKFDQAPKVDAKFDNAAMDDFAQYSAWAEIEALPNLTEMQKDQFIWSLMFDPSVSKEVLGEAQKLNDSQAPKQAPKVDAKFDQAPKVDAKFDNAAMDDFAQYSAWAEIEALPNLTEMQKDQFIWSLMFDPSVSKEVLGEAQKLNDSQAPKQAPKVDAKFDQAPKVDAKFDNAAMDDFAQYSAWAEIEALPNLTEMQKDQFIWSLMFDPSVSKEVLGEAQKLNDSQAPKC

(19) United States
(12) Patent Application Publication
(10) Pub. No.: US 2024/0309056 A1
(43) Pub. Date: Sep. 19, 2024

(54) Affinity ligands for the purification and detection of ubiquitin muteins

(71) Applicant: Navigo Proteins GmbH, Halle/Saale (DE)

(72) Inventors: Erik Fiedler, Halle/Saale (DE); Mathias Kahl, Halle/Saale (DE); Anja Katzchmann, Halle/Saale (DE); Florian Settele, Halle/Saale (DE)

(73) Assignee: Navigo Proteins GmbH, Halle/Saale (DE)

(21) Appl. No.: 18/575,743

(22) PCT Filed: Jun. 30, 2022
(86) PCT No.: PCT/EP2022/068207

§ 371 Date: Dec. 29, 2023

(30) Foreign Application Priority Data
Jul. 13, 2021 (EP) 21185395.7

(51) Int. Cl. C07K 14/31; C07K 1/22; G01N 33/68

(52) U.S. Cl. CPC C07K 14/31; C07K 1/22; G01N 33/68; C07K 2319/705

(57) ABSTRACT

The present invention relates to novel affinity ligands for the affinity purification of ubiquitin muteins. In particular, the alkaline stable affinity ligands are useful for affinity chromatography purification and detection of a ubiquitin mutein (such as Affilin® protein). The novel affinity ligands for a ubiquitin mutein are based on artificial mosaic proteins of Protein A-like structure. The affinity ligands of the invention can be used for the purification and detection of ubiquitin muteins.

### TECHNICAL FIELD

The present invention relates to novel affinity ligands for the affinity purification. In particular, the alkaline stable affinity ligands are useful for affinity chromatography purification and detection of a ubiquitin mutein (such as Affilin® protein). The novel affinity ligands for a ubiquitin mutein are based on artificial mosaic proteins of Protein A-like structure. The affinity ligands of the invention can be used for the efficient purification and detection of monomeric or dimeric ubiquitin muteins.

### BACKGROUND OF THE INVENTION

Ubiquitin muteins, even modular or multimeric versions can routinely be produced as single chain proteins with good yields and purity, rapidly and cost-effectively in standard E. coli expression systems. However, current purification process of ubiquitin muteins (for example, Affilin® proteins) involve generally several purification steps. For example, one common purification method of ubiquitin muteins is a three-step chromatography purification including hydrophobic interaction, ion exchange chromatography (IEC), and size exclusion chromatography (SEC). Such three-step procedure might not be suitable for all Affilin® molecules. Further, there is currently no efficient and reliable detection method for Affilin® proteins in solutions available.

There is a strong need to provide methods for a more simple and efficient one-step purification and detection of ubiquitin muteins. The present invention meets this need by providing novel affinity ligands for ubiquitin muteins. This will open successful purification and detection of ubiquitin muteins, for example, for quantities economically viable for technical or medical purposes.

The above overview does not necessarily describe all problems solved by the present invention.

### SUMMARY OF THE INVENTION

The present disclosure provides the following items 1 to 15, without being specifically limited thereto:
1. An affinity ligand comprising an amino acid sequence with at least 90% sequence identity to SEQ ID NO: 5, preferably wherein the affinity ligand is comprising an amino acid sequence with at least 90% sequence identity to SEQ ID NO: 1, 2, 3, 4, or 21, preferably wherein the affinity ligand is stable under alkaline conditions of 0.1 M NaOH incubation for at least 6 h, more preferably wherein the affinity has a remaining DBC10 of at least 88% after 0.1 M NaOH incubation for at least 6 h. The affinity ligand may comprise an amino acid sequence with at least 90% sequence identity to at least 56 amino acids of SEQ ID NO: 1, wherein the affinity ligand is stable under alkaline conditions of 0.1 M NaOH incubation for at least 6 h, and wherein the affinity ligand has a binding affinity of less than 100 nM for a ubiquitin mutein, preferably wherein the affinity has a remaining DBC10 of at least 88% after 0.1 M NaOH incubation for at least 6 h.
2. The affinity ligand according to item 1 wherein the affinity ligand has a binding affinity of less than 500 nM, preferably less than 100 nM for a ubiquitin mutein.
3. The affinity ligand according to item 1 wherein the affinity ligand binds to a ubiquitin mutein having at least 80% identity to ubiquitin of SEQ ID NO: 20.
4. The affinity ligand according to item 1 wherein the ubiquitin mutein is a monomer or a dimer.
5. The affinity ligand according to item 4 wherein a ubiquitin mutein is selected from amino acid sequences with at least 80% identity to the group of SEQ ID NOs: 10-19.
6. The affinity ligand according to items 1-5 wherein the affinity ligand is fused to at least one non-Immunoglobulin-binding protein.
7. The affinity ligand according to item 6 wherein the affinity ligand is fused to at least one non-Immunoglobulin-binding protein with at least 90% identity to SEQ ID NO: 9, preferably wherein the affinity ligand is fused to one, two, or three non-Immunoglobulin-binding protein(s) with at least 90% identity to SEQ ID NO: 9.
8. The affinity ligand according to any one of item 7 for use in affinity purification of a ubiquitin mutein or a protein comprising a ubiquitin mutein.
9. An affinity separation matrix comprising an affinity ligand according to any one of items 1-7
10. Use of the affinity ligand according to any one of items 1-7, or the affinity separation matrix according to item 9 for affinity purification of a ubiquitin mutein or a protein comprising a ubiquitin mutein.
11. A process of manufacturing a ubiquitin mutein comprising at least one chromatographic step employing an affinity chromatography matrix having an affinity for a ubiquitin mutein wherein the affinity ligand according to any one of items 1-7 is coupled to said affinity chromatography matrix.
12. A method of affinity purification of a ubiquitin mutein, the method comprising:
   (a) providing a liquid that contains a ubiquitin mutein, preferably a ubiquitin mutein with at least 80% identity to SEQ ID NO: 20;
   (b) providing an affinity separation matrix comprising at least one affinity ligand according to any one of items 1-7 coupled to said affinity separation matrix;
   (c) contacting said affinity separation matrix with the liquid under conditions that permit binding of the at least one affinity ligand according to any one of items 1-7; and
   (d) eluting said ubiquitin mutein from said affinity purification matrix, thereby obtaining an eluate containing said ubiquitin mutein.
13. A method according to claim 12 wherein in step (d) the elution pH is 3.5 or higher, preferably wherein the elution pH is between 3.5 and 4.5.
14. A method of analyzing the presence of a ubiquitin mutein in liquid samples, the method comprising the following steps:
   (i) providing a liquid that contains a ubiquitin mutein,
   (ii) providing the affinity ligand for a ubiquitin mutein according to items 1-7,
   (iii) contacting the liquid of (i) with the affinity ligand according to items 1-7 under conditions that permit binding of the affinity ligand to the ubiquitin mutein, and
   (iv) determining the amount of the affinity ligand according to items 1-7 in the liquid of (i).
15. A polynucleotide encoding the affinity ligand according to any one of items 1-7.

This summary of the invention is not limiting, and other aspects and embodiments of the invention will become evident from the following description, examples and drawings.

### BRIEF DESCRIPTION OF THE FIGURES

FIG. 1: Amino acid sequences of affinity ligands for ubiquitin muteins (Affilin® proteins).

FIG. 2: SPR analysis of the affinity binding of ubiquitin mutein 77404 to fusion protein 214410

FIG. 3: Caustic stability of affinity ligands (as fusion proteins 214410, 214411, and 214412, respectively). Shown is the UV280 nm breakthrough profile. The remaining DBC 10% after 0.1 M NaOH incubation was at least 88% for 214410, at least 89% for 214412, and at least 95% for 214411.

FIG. 4. shows the SDS-PAGE of ubiquitin mutein 77404 purified via a one-step purification affinity chromatography with Praesto_214411 (resin with immobilized fusion protein comprising SEQ ID NO: 2). E. coli lysate (2.5 g BWM of ubiquitin mutein 77404) purified via Praesto_214411 was analyzed with SDS-PAGE. Different amounts of neutralized elution fractions were applied. Ubiquitin mutein 77404 was eluted with acid pH 7.0-2.0. No significant impurities were detected. Lane 1: PageRuler Unstained Protein Ladder, lane 2: pellet, lane 3: supernatant, lane 4: flow-through; lanes 5-7: eluted fractions.

### DETAILED DESCRIPTION OF THE INVENTION

The present invention provides novel affinity ligands with high alkaline stability. The novel proteins of the present invention are particularly advantageous because they allow precise purification of diverse ubiquitin muteins or ubiquitin via the one-step method of affinity chromatography. Further, the affinity ligands of the invention allow detection and analysis of diverse ubiquitin muteins or ubiquitin. Any polypeptide of SEQ ID NO: 5, preferably selected from the group of SEQ ID NOS: 1-4, or an amino acid sequence with at least 90% identity to SEQ ID NO: 5, preferably to any one of SEQ ID NOs: 1-4, 21, is alkaline stable and binds to proteins with at least 80% identity to ubiquitin. Thus, the proteins of the invention can be used as affinity ligand for ubiquitin or ubiquitin muteins, for example, for a one step purification or detection method.

Before the present invention is described in more detail below it is to be understood that this invention is not limited to the particular methodology, protocols and reagents described herein as these may vary. It is also to be understood that the terminology used herein is for the purpose of describing particular aspects and embodiments only and is not intended to limit the scope of the present invention, which is reflected by the appended items. Unless defined otherwise, all technical and scientific terms used herein have the same meaning as commonly understood by one of ordinary skill in the art to which this invention belongs. This includes a skilled person working in the field of protein purification and detection and analysis, but also including a skilled person working in the field of developing new specific ligands for ubiquitin muteins for use in technical applications, for example for use as affinity ligands in affinity chromatography.

Preferably, the terms used herein are defined as described in “A multilingual glossary of biotechnological terms: (IUPAC Recommendations)”, Leuenberger, H.G.W, Nagel, B. and Kölbl, H. eds. (1995), Helvetica Chimica Acta, CH-4010 Basel, Switzerland).

Throughout this specification and the items, which follow, unless the context requires otherwise, the word “comprise”, and variants such as “comprises” and “comprising”, will be understood to imply the inclusion of a stated integer or step, or group of integers or steps, but not the exclusion of any other integer or step or group of integers or steps. The term “comprise(s)” or “comprising” may encompass a limitation to “consists of” or “consisting of”, should such a limitation be necessary for any reason and to any extent.

Several documents (for example: patents, patent applications, scientific publications, manufacturer's specifications, instructions, GenBank Accession Number, etc.) may be cited throughout the present specification. Nothing herein is to be construed as an admission that the invention is not entitled to antedate such disclosure by virtue of prior invention. Some of the documents cited herein may be characterized as being “incorporated by reference”. In the event of a conflict between the definitions or teachings of such incorporated references and definitions or teachings recited in the present specification, the text of the present specification takes precedence.

All sequences referred to herein are disclosed in the attached sequence listing that, with its whole content and disclosure, forms part of the disclosure content of the present specification.

### General Definitions of Important Terms Used in the Application

The term “ubiquitin mutein” or “Affilin® protein” relates to modified ubiquitin protein. Ubiquitin is shown in SEQ ID NO: 20. The term “ubiquitin mutein” refers to an amino acid sequence as shown in SEQ ID NOs: 10-19. The term “ubiquitin mutein” comprises all polypeptides which show an amino acid sequence identity of at least 70%, 80%, 85%, 90%, 95%, 96% or 97% or more, or 100% to SEQ ID Nos: 10-19. A ubiquitin mutein is a monomer or can be comprised of two monomers resulting in a dimer. A ubiquitin mutein is specific for a defined target, for example but not limited to, a cancer related target. In various embodiments of the invention, the term “ubiquitin mutein” refers to an amino acid sequence exhibiting 80% to 94% identity to ubiquitin (SEQ ID NO: 20). In various other embodiments of the invention, the term “ubiquitin mutein” refers to an amino acid sequence exhibiting at least 85% and up to 94% identity to ubiquitin (SEQ ID NO: 20).

The term “affinity ligand” describes a protein that is capable to bind to a ubiquitin mutein, as defined above. As described herein, an affinity ligand refers to a protein with detectable interaction with a ubiquitin mutein, as determined by suitable methods such as for example SPR analysis or BLI or other appropriate technology known to someone skilled in the art.

The terms “binding affinity” and “binding activity” may be used herein interchangeably and they refer to the ability of a polypeptide of the invention to bind to ubiquitin muteins including a fragment or domain thereof. Binding affinity is typically measured and reported by the equilibrium dissociation constant (K_{D}) which is used to evaluate and rank the strength of bimolecular interactions. The binding affinity and dissociation constants can be measured quantitatively. Methods for determining binding affinities are well known to the skilled person and can be selected, for instance, from the following methods that are well established in the art: surface plasmon resonance (SPR), Bio-layer interferometry (BLI), enzyme-linked immunosorbent assay (ELISA), kinetic exclusion analysis (KinExA assay), flow cytometry, fluorescence spectroscopy techniques, isothermal titration calorimetry (ITC), analytical ultracentrifugation, radioimmunoassay (RIA or IRMA), and enhanced chemiluminescence (ECL). Typically, the dissociation constant K_{D} is determined at temperatures in the range of 20° C. and 30° C. If not specifically indicated otherwise, K_{D} values recited herein are determined at 25° C. by SPR. The most widely used SPR-based system is the BIAcore, produced by BIAcore AB. In various embodiments of the present invention, the binding affinity for ubiquitin muteins may be determined by the BIAcore SPR system.

The term “fusion protein” relates to a protein comprising at least a first protein joined genetically to at least a second protein. A fusion protein is created through joining of two or more genes that originally coded for separate proteins. Thus, a fusion protein may comprise a multimer of identical or different proteins which are expressed as a single, linear polypeptide. For example, an affinity ligand of the invention is fused to 1, 2, 3 or more non-Ig-binding protein(s) to generate a fusion protein that can be used for affinity purification of ubiquitin muteins.

The term “fused” means that the components are linked by peptide bonds, either directly or via peptide linkers.

As used herein, the term “linker” refers in its broadest meaning to a molecule that covalently joins at least two other molecules. In preferred embodiments, the linker is a peptide linker, i.e. the moiety linking the two proteins is one single amino acid or a peptide comprising two or more amino acids. In one embodiment of the present invention, a linker is to be understood as a moiety that connects an ubiquitin mutein binding domain with at least one further ubiquitin ubiquitin binding domain, i.e. a moiety linking two proteins to each other to generate a multimer. In one embodiment of the present invention, a linker is to be understood as a moiety that connects an ubiquitin mutein binding domain with at least one, two, or three non-Ig binding domain(s). In one embodiment of the present invention, a linker is to be understood as a moiety that connects an non-Ig binding domain with another non-Ig binding domain(s).

In various preferred embodiments, a linker as described above may be a peptide linker, more specifically a Gly-Ser linker. In preferred embodiments, the linker may be a G4S or a (G4S)2 or a (G4S)3 linker, preferably a (G4S)2 linker.

The term “amino acid sequence identity” refers to a quantitative comparison of the identity (or differences) of the amino acid sequences of two or more proteins. “Percent (%) amino acid sequence identity” with respect to a reference polypeptide sequence is defined as the percentage of amino acid residues in a sequence that are identical with the amino acid residues in the reference polypeptide sequence, after aligning the sequences and introducing gaps, if necessary, to achieve the maximum percent sequence identity. To determine the sequence identity, the sequence of a query protein is aligned to the sequence of a reference protein or polypeptide, for example, to the polypeptide of SEQ ID NO: 1. Methods for sequence alignment are well known in the art. For example, for determining the extent of an amino acid sequence identity of an arbitrary polypeptide relative to the amino acid sequence of, for example, SEQ ID NO: 1, the SIM Local similarity program is preferably employed (Xiaoquin Huang and Webb Miller (1991), Advances in Applied Mathematics, vol. 12: 337-357), that is freely available. For multiple alignment analysis, ClustalW is preferably used (Thompson et al. (1994) Nucleic Acids Res., 22(22): 4673-4680).

The terms “protein” and “polypeptide” refer to any chain of two or more amino acids linked by peptide bonds and does not refer to a specific length of the product. Thus, “peptides”, “protein”, “amino acid chain”, or any other term used to refer to a chain of two or more amino acids, are included within the definition of “polypeptide”, and the term “polypeptide” may be used instead of, or interchangeably with, any of these terms. The term “polypeptide” is also intended to refer to the products of post-translational modifications of the polypeptide like, e.g., glycosylation, which are well known in the art.

The term “alkaline stable” or “alkaline stability” or “caustic stable” or “caustic stability” refers to the ability of the affinity ligand to withstand alkaline conditions without significantly losing the ability to bind ubiquitin or a ubiquitin mutein. The skilled person in this field can easily test alkaline stability by incubating a ubiquitin mutein with sodium hydroxide solutions, e.g., as described in the Examples, and subsequent testing of the binding activity to ubiquitin mutein thereof by routine experiments known to someone skilled in the art, for example, by chromatographic approaches.

The term “chromatography” refers to separation technologies which employ a mobile phase and a stationary phase to separate one type of molecules (e.g., ubiquitin mutein) from other molecules (e.g., contaminants) in the sample. The liquid mobile phase contains a mixture of molecules and transports these across or through a stationary phase (such as a solid matrix). Due to the differential interaction of the different molecules in the mobile phase with the stationary phase, molecules in the mobile phase can be separated.

The term “affinity chromatography” refers to a specific mode of chromatography in which a ligand (i.e. an affinity ligand) coupled to a stationary phase interacts with a molecule (i.e. ubiquitin mutein) in the mobile phase (the sample) i.e. the ligand has a specific binding affinity for the molecule to be purified. As understood in the context of the invention, affinity chromatography involves the addition of a sample containing a ubiquitin mutein to a stationary phase which comprises a chromatography ligand, such as an affinity ligand. The terms “solid support” or “solid matrix” are used interchangeably for the stationary phase.

The terms “affinity matrix” or “affinity purification matrix” or “affinity chromatography matrix”, as used interchangeably herein, refer to a matrix, e.g., a chromatographic matrix, onto which an affinity ligand e.g., an affinity ligand is attached. The attached affinity ligand (e.g., affinity ligand) is capable of specific binding to a molecule of interest (e.g., ubiquitin mutein) which is to be purified or removed from a mixture.

The term “affinity purification” as used herein refers to a method of purifying a ubiquitin mutein from a liquid by binding ubiquitin muteins to affinity ligand that is immobilized to a matrix. Thereby, other components of the mixture except ubiquitin muteins are removed. In a further step, the bound protein can be eluted in highly purified form.

### Detailed Description of the Embodiments of the Invention

The present invention will now be further described. In the following passages different aspects of the invention are defined in more detail. Each aspect defined below may be combined with any other aspect or aspects unless clearly indicated to the contrary. In particular, any feature indicated as being preferred or advantageous may be combined with any other feature or features indicated as being preferred or advantageous.

One advantage of the disclosed affinity ligand is the important functional characteristic that it is stable under alkaline conditions of 0.1 M NaOH incubation for at least 6 h, more preferably wherein the affinity has a remaining DBC10 of at least 88% after 0.1 M NaOH incubation for at least 6 h. In addition, the affinity ligand binds to ubiquitin muteins. Needless to point out, that this is of particular advantage in the purification and/or detection and/or analysis of ubiquitin muteins.

The general scaffold of the affinity ligand of the invention is an artificial mosaic protein composed of fragments of several wild-type Protein A (SpA) domains having further specific mutations that generate the unique and surprising functionality. The novel affinity ligands of the invention are alkaline stable and exhibit a binding affinity for ubiquitin muteins.

In some embodiments, the affinity ligand for ubiquitin mutein (Affilin®) proteins is comprising an amino acid sequence with at least 90% sequence identity to at least 56 amino acids of SEQ ID NO: 5:

NAAMDDFX₁QYSX₂AWAEIEALPNLTEMQKDQFIWSLMFDPSVSX₃EX₄
LX₅EAX₆KLNDX₇QAPK

wherein position X₁ is selected from any amino acid, preferably from A or D,
wherein position X₂ is selected from any amino acid, preferably from S or A,
wherein position X₃ is selected from any amino acid, preferably from K or L,
wherein position X₄ is selected from any amino acid, preferably from V or I,
wherein position X₅ is selected from any amino acid, preferably from G or A,
wherein position X₆ is selected from any amino acid, preferably from Q or K, and
wherein position X₇ is selected from any amino acid, preferably from S or A.

In further embodiments, the amino acids at positions selected from any of positions 10, 13, 14, 17, 18, 24, 25, 27, 28, 29, 32, 35, and 36 are any of 10Y, 13W, 14A, 17E, 18A, 24E, 25M, 27K, 28D, 29Q, 32W, 35M, and/or 36F.

L=Leucine (Leu), A=Alanine (Ala), E=Glutamic acid (Glu), K=Lysine (Lys), V=Valine (Val), D=Aspartic acid (Asp), G=Glycine (Gly), S=Serine (Ser), Y=Tyrosine (Tyr), I=Isoleucine (Ile), T=Threonine (Thr), N=Asparagine (Asn), F=Phenylalanine (Phe), R=Arginine (Arg), Q=Glutamine (Gln), P=Proline (Pro), M=Methionine (Met), W=Tryptophan (W), H=Histidine (His), C=Cysteine (C).

In some embodiments, the binding protein is comprising an amino acid sequence with at least 90% sequence identity to at least 56 amino acids of SEQ ID NO: 1:

NAAMDDFAQYSAWAEIEALPNLTEMQKDQFIWSLMFDPSVSKEVLGEAQK
LNDSQAPK.

The affinity ligands of the invention differ in amino acids positions 8, 11, 42, 44, 46, 49, and 53 in SEQ ID NO: 1 or SEQ ID NO: 3. Position 8 corresponding to position 8 in SEQ ID NO: 1 or SEQ ID NO: 3 is A or D. Position 11 corresponding to position 11 in SEQ ID NO: 1 or SEQ ID NO: 3 is S or A. Position 42 corresponding to position 42 in SEQ ID NO: 1 or SEQ ID NO: 3 is K or L. Position 44 corresponding to position 44 in SEQ ID NO: 1 or SEQ ID NO: 3 is V or I. Position 46 corresponding to position 46 in SEQ ID NO: 1 or SEQ ID NO: 3 is G or A. Position 49 corresponding to position 49 in SEQ ID NO: 1 or SEQ ID NO: 3 is Q or K. Position 53 corresponding to position 53 in SEQ ID NO: 1 or SEQ ID NO: 3 is S or A. SEQ ID NO: 1 and SEQ NO: 3 have 90.8% amino acid identity in 58 amino acids.

In various embodiments, the affinity ligand has a deletion in the amino acids corresponding to the amino acids at positions 1 and 2 of SEQ ID NOs: 1, 3, 5, and 21. SEQ ID NO: 2 is the same as SEQ ID NO: 1 but two N-terminal amino acids are deleted. SEQ ID NO: 4 is the same as SEQ ID NO: 3 but two N-terminal amino acids are deleted.

In various embodiments, in the affinity ligands of the invention, the amino acid at the positions corresponding to positions 4, 5, 7, 10, 13, 14, 17, 18, 24, 25, 27, 28, 29, 32, 35, and 36, in SEQ ID NO: 5 is not K, F, E, Q, F, Y, L, H, E, E, R, N, A, Q, R, and D, respectively. In various embodiments, in the affinity ligands of the invention, the amino acid at the positions corresponding to positions 4, 5, 7, 10, 13, 14, 17, 18, 24, 25, 27, 28, 29, 32, 35, and 36, in SEQ ID NO: 5 is M, D, F, Y, W, A, E, A, E, M, D, Q, W, M, and F, respectively. In various embodiments, in the affinity ligands of the invention, the amino acid at the positions corresponding to positions 4, 5, 7, 10, 13, 14, 17, 18, 24, 25, 27, 28, 29, 32, 35, and 36, in SEQ ID NO: 5 is R, S, E, Y, W, A, E, A, E, M, D, Q, W, M, and F, respectively.

In some embodiments, in the affinity ligands of the invention, the amino acid at the positions corresponding to positions 8, 11, 42, 44, 46, 49, and 54 in SEQ ID NO: 5 is A, S, K, V, G, Q, and S, respectively. In some embodiments, in affinity ligands of the invention, the amino acid at the positions corresponding to positions 8, 11, 42, 44, 46, 49, and 54, in SEQ ID NO: 5 is not A, S, K, V, G, Q, and S, respectively. In other embodiments, in the affinity ligands of the invention, the amino acid at the positions corresponding to positions 8, 11, 42, 44, 46, 49, and 54 in SEQ ID NO: 5 is D, A, L, I, A, K, and A, respectively.

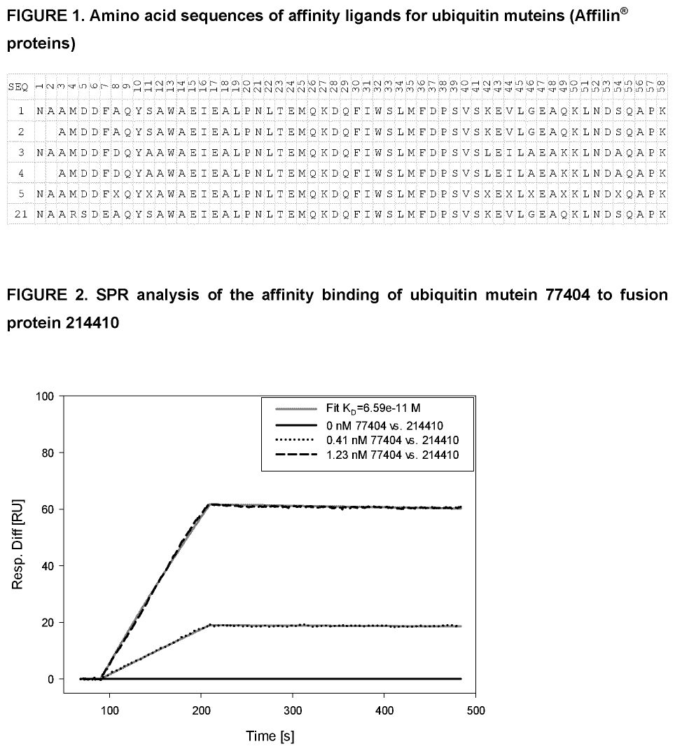

The affinity ligand comprises the amino acid sequence selected from the group of SEQ ID NOs: 1-5, 21 or an amino acid with at least 90% sequence identity to any one of SEQ ID NOs: 1-5, 21. In some embodiments, an affinity ligand is comprising at least one amino acid sequence as shown in FIG. 1.

In some embodiments defined herein, the affinity ligand has at least 90% sequence identity, the sequence identity may preferably be any of at least 91%, 92%, 93%, 94%, 95%, 96%, 97%, 98%, 99%, or 100% sequence identity to any one of the amino sequences of SEQ ID NOs: 1-5, 21.

The affinity ligand binds to ubiquitin and to ubiquitin muteins having at least 80% identity to ubiquitin (SEQ ID NO: 20). In various embodiments, the affinity ligand binds to ubiquitin and to ubiquitin muteins having between 80% and 90% identity to ubiquitin (SEQ ID NO: 20). Ubiquitin muteins may be monomers or dimers. Optionally to 6-15 substitutions in SEQ ID NO: 20, the ubiquitin mutein has an additional insertion of 4-8 amino acids, preferably between position 9-10.

In some embodiments, the affinity ligand of the invention binds to SEQ ID NOs: 14-16 (Affilin®-77404). SEQ ID NO: 14 is a dimer of two ubiquitin muteins (SEQ ID NO: 15 and SEQ ID NO: 16), each monomer having 8 or 9 different amino acids compared to SEQ ID NO: 20, having 88.1% or 89.5% identity, respectively, to ubiquitin of SEQ ID NO: 20.

In some embodiments, the affinity ligand binds to SEQ ID NOs: 17-20 (Affilin®-200053). SEQ ID NO: 17 is a dimer of two ubiquitin muteins (SEQ ID NO: 18 and SEQ ID NO: 20), each mutein having 8 different amino acids compared to SEQ ID NO: 20, having 88.1% identity to ubiquitin of SEQ ID NO: 20. In some embodiments, the affinity ligand binds to SEQ ID NOs: 11 (Affilin®-139819).

SEQ ID NO: 10 (Affilin®-211883) has 10 different amino acids compared to SEQ ID NO: 20, thus having 86.8% identity to ubiquitin of SEQ ID NO: 20.

SEQ ID NO: 11 (Affilin®-139819) has 6 different amino acids compared to SEQ ID NO: 20 and an additional insertion of 6 amino acids between position 9 and position 10 of SEQ ID NO: 20, thus having 84% identity to ubiquitin of SEQ ID NO: 20.

SEQ ID NO: 12 (Affilin®-215208) has 9 different amino acids compared to SEQ ID NO: 20 and an additional insertion of 4 amino acids between position 9 and position 10 of SEQ ID NO: 20, thus having 82.9% identity to ubiquitin of SEQ ID NO: 20.

SEQ ID NO: 13 (Affilin®-215220) has 9 different amino acids compared to SEQ ID NO: 20 and an insertion of 6 amino acids between position 9 and position 10 of SEQ ID NO: 20, thus having 80.2% identity to ubiquitin of SEQ ID NO: 20.

Multimers. In one embodiment of the invention, the affinity ligand comprises 1, 2, 3, 4, 5, or 6 affinity ligand(s) linked to each other. In one embodiment, the ligand comprises 2, 3, or 4 affinity ligands linked to each other, either directly or via a peptide linker. Multimers of the affinity ligand are generated artificially, generally by recombinant DNA technology well-known to a skilled person. In some embodiments, the multimer is a homo-multimer, e.g. the amino acid sequences of affinity ligand are identical. Examples are provided for homo-multimers of SEQ ID NO: 1 or of SEQ ID NO: 21. In one embodiment, the ligand comprises 2, 3, 4 affinity ligand(s) linked to each other. A dimer of SEQ ID NO: 1 is provided in SEQ ID NO: 26 (216599). A trimer of SEQ ID NO: 1 is provided in SEQ ID NO: 27 (216000). A tetramer of SEQ ID NO: 1 is provided in SEQ ID NO: 28 (216001). A dimer of SEQ ID NO: 21 is provided in SEQ ID NO: 23 (216596). A trimer of SEQ ID NO: 21 is provided in SEQ ID NO: 24 (216597). A tetramer of SEQ ID NO: 21 is provided in SEQ ID NO: 25 (216598).

In other embodiments, the multimer is a hetero-multimer, e.g. the amino acid sequences of the affinity ligand are different.

Fusion proteins. In some embodiments, the affinity ligand as described above is fused to at least one further polypeptide distinct from the polypeptide as disclosed. In various embodiments, the further polypeptide distinct from the affinity ligand as disclosed herein might be a non-Immunoglobulin binding protein, for example but not limited to, a protein that does not bind to the Fc part of immunoglobulin (Ig). In some embodiments, a non-Ig binding protein has at least 89.5% identity to SEQ ID NO: 9. In various embodiments, a non-Ig binding protein has at least 89.5% identity to SEQ ID NO: 9, and has a D or E, preferably a D, at the position corresponding to position 13 of SEQ ID NO: 9, and/or has an R, K, or H, preferably R, at the position corresponding to position 31 of SEQ ID NO: 9. In various embodiments, the non-Ig binding protein has a S at one or more of the positions corresponding to positions 10 and 14 of SEQ ID NO: 9. In various embodiments, the non-Ig binding protein may have a Q at the position corresponding to position 10 of SEQ ID NO: 9, and/or may have a K at the position corresponding to position 14 of SEQ ID NO: 9. In various embodiments, the non-Ig binding protein may have an I at the position corresponding to position 8 of SEQ ID NO: 9.

A fusion of the binding protein with one, two, three or more non-Ig binding protein(s) may improve expression of the protein and ligand detectability in the Protein A ELISA leaching assay. The non-Ig-binding protein has no detectable binding affinity for the Fc domain of immunoglobulin as determined by SPR spectroscopy, more specifically the BIAcore SPR system. Further, non-Ig-binding protein has no detectable binding affinity for the ubiquitin or ubiquitin muteins, as determined by SPR spectroscopy, more specifically the BIAcore SPR system.

Accordingly, some embodiments encompass fusion proteins comprising an affinity ligand as disclosed herein and one or two or three or more non-Ig-binding polypeptide(s). In some embodiments, fusion proteins comprising at least one affinity ligand and three non-Ig binding protein are provided in SEQ ID NOs: 6-8. The non-Ig binding proteins may be the same or different in the fusion protein.

The present invention encompasses fusion proteins comprising an affinity ligand having at least 80% sequence identity to SEQ ID NO: 5, preferably at least 80% or at least 90% identity to SEQ ID NOs: 1-4, and at least one non-Ig binding protein having at least 89.5% identity to SEQ ID NO: 9. In preferred embodiments, the affinity ligand is located at the N-terminus of the fusion protein. In particular, the present invention encompasses fusion proteins comprising a ligand at least 80% sequence identity to SEQ ID NO: 5, preferably at least 80% or at least 90% identity to SEQ ID NOs: 1-4, and three non-Ig bindings protein having at least 89.5% identity to SEQ ID NO: 9. In preferred embodiments, affinity ligand is located at the N-terminus of the fusion protein.

For example, SEQ ID NO: 6 (214410) is a fusion protein wherein the affinity ligand of SEQ ID NO: 1 (187029) is fused to a trimer of non-Ig binding protein according to SEQ ID NO: 9.

For example, SEQ ID NO:7 (214411) is a fusion protein wherein the affinity ligand of SEQ ID NO: 2 (187029 del2N) is fused to a trimer of non-Ig binding protein according to SEQ ID NO: 9.

For example, SEQ ID NO: 8 (214412) is a fusion protein wherein the affinity ligand of SEQ ID NO: 3 is fused to a trimer of non-Ig binding protein according to SEQ ID NO: 9.

For example, SEQ ID NO: 22 (220075) is a fusion protein wherein the affinity ligand of SEQ ID NO: 2 and SEQ ID NO: 1 is fused to a dimer of non-Ig binding protein according to SEQ ID NO: 9. The fusion protein comprises a peptide linker of 20 amino acids between SEQ ID NO: 2 and SEQ ID NO: 1. SEQ ID NO: 2 is in 56 amino acids identical to SEQ ID NO: 1.

In various embodiments, the fusion protein has a binding affinity of less than 100 nM, preferably less than 50 nM, more preferably less than 10 nM for ubiquitin or ubiquitin muteins.

Linker. In some embodiments of the first aspect, the one or more binding domain(s) are directly linked to each other. In other embodiments, the one or more binding domains are linked to each other with one or more linkers, preferably peptide linkers. The peptide linker is connected to the first protein and to the second protein by a peptide bond between the C-terminal and N-terminal ends of the domains, thereby generating a single, linear polypeptide chain (fusion protein). The length and composition of a linker may vary between at least one and up to about 30 amino acids. More specifically, a peptide linker has a length of between 1 and 30 amino acids; e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 amino acids. It is preferred that the amino acid sequence of the peptide linker is stable against caustic conditions and proteases. Linkers should not destabilize the conformation of the protein.

Molecules for purification or detection. In some embodiments, the affinity ligand may also comprise additional amino acid residues at the N- and/or C-terminal end, such as for example an additional sequence at the N- and/or C-terminal end. Additional sequences may include for example sequences introduced e.g. for purification or detection. Typical examples for such sequences include, without being limiting, Strep-tags, oligohistidine-tags, glutathione S-transferase, maltose-affinity ligand, inteins, intein fragments, or the albumin-binding domain of protein G, or others. In one embodiment, additional amino acid sequences include one or more peptide sequences that confer an affinity to certain chromatography column materials. The affinity ligand may include specific attachment sites for the attachment to solid supports, preferably at the C-terminal end, such as cysteine or lysine.

Use of the novel affinity ligand in technical applications. Also provided herein is the use of any novel affinity ligand as disclosed herein, including multimers, including fusion proteins, in technical applications, preferably for use in affinity purification.

As described herein, affinity chromatography (also called affinity purification) makes use of specific binding interactions between molecules. Methods for immobilization of protein and methods for affinity chromatography are well-known in the field of protein purification and can be easily performed by a skilled person in this field using standard techniques and equipment. In various embodiments, the method of affinity purification may further comprise one or more washing steps carried out under conditions sufficient to remove from the affinity purification matrix some or all molecules that are non-specifically bound thereto. Affinity purification matrices suitable for the disclosed uses and methods are known to a person skilled in the art.

Conjugation to a solid support. In various aspects and/or embodiments of the present invention, the novel proteins disclosed herein including novel proteins generated or obtained by any of the methods as described above are conjugated to a solid support. In some embodiments of the invention, the polypeptide comprises an attachment site for site-specific covalent coupling of the polypeptide to a solid support. Specific attachment sites comprise without being limited thereto, natural amino acids, such as cysteine or lysine, which enable specific chemical reactions with a reactive group of the solid phase, or a linker between the solid phase and the protein.

Affinity purification matrix. In another embodiment, an affinity purification matrix is provided comprising an affinity ligand for ubiquitin muteins, including a polypeptide identified by any of the methods as described above.

In preferred embodiments, the affinity purification matrix is a solid support. The affinity purification matrix comprises at least one affinity ligand as described herein. Accordingly, a novel affinity ligand disclosed herein is encompassed for use in the purification of ubiquitin muteins by an affinity matrix.

Solid support matrices for affinity chromatography are known in the art and include, e.g., without being limited thereto, agarose and stabilized derivatives of agarose, cellulose or derivatives of cellulose, controlled pore glass, monolith, silica, zirconium oxide, titanium oxide, or synthetic polymers, and hydrogels of various compositions and combinations of the above. The formats for solid support matrices can be of any suitable well-known kind. Such solid support matrix for coupling a novel protein or polypeptide of the present invention might comprise, e.g., one of the following, without being limited thereto: columns, capillaries, particles, membranes, filters, monoliths, fibers, pads, gels, slides, plates, cassettes, or any other format commonly used in chromatography and known to someone skilled in the art.

In one embodiment, the matrix is comprised of substantially spherical beads, for example Sepharose or Agarose beads. Matrices in particle form can be used as a packed bed or in a suspended form including expanded beds. In other embodiments of the invention, the solid support matrix is a membrane, for example a hydrogel membrane. In some embodiments, the affinity purification may involve a membrane as a matrix to which an affinity ligand of the present invention is covalently bound. The solid support can also be in the form of a membrane in a cartridge.

In some embodiments, the affinity purification involves a chromatography column containing a solid support matrix to which a novel protein of the present invention is covalently bound. A novel protein or polypeptide of the present invention may be attached to a suitable solid support matrix via conventional coupling techniques. Methods for immobilization of protein ligands to solid supports are well-known in the field of protein engineering and purification and can easily be performed by a skilled person in this field using standard techniques and equipment.

Method of purification. Further embodiments relate to a process of manufacturing ubiquitin muteins comprising at least one chromatographic step employing an affinity chromatography matrix having an affinity for binding ubiquitin muteins wherein the affinity ligand as described above is coupled to said affinity chromatography matrix. Various embodiments relate to a method of purifying ubiquitin muteins via one chromatographic step employing the affinity ligand of the invention immobilized to an affinity chromatography matrix as described above. In some embodiments, the method of affinity purification of a ubiquitin mutein is comprising: (a) providing a liquid that contains any one of ubiquitin muteins of SEQ ID NOs: 10-19, or of muteins with at least 80% identity thereto, respectively; (b) providing an affinity separation matrix comprising at least the affinity ligand of the invention coupled to said affinity separation matrix; (c) contacting said affinity separation matrix with the liquid under conditions that permit binding of the ubiquitin mutein; and (d) eluting said ubiquitin mutein from said affinity purification matrix, preferably with elution pH of 3.5, preferably with elution pH in the range of 3.5-4.5, thereby obtaining an eluate containing said ubiquitin mutein with purity of at least 83%, preferably at least 90%, more preferably 100%, as determined via SDS-PAGE.

In some embodiments, the method of affinity purification of ubiquitin mutein of SEQ ID NO: 14 is comprising: (a) providing a liquid that ubiquitin muteins of SEQ ID NO: 14, or of muteins with at least 80% identity thereto; (b) providing an affinity separation matrix comprising a fusion protein comprising the affinity ligand of SEQ ID NO: 1-5, 21 coupled to said affinity separation matrix; (c) contacting said affinity separation matrix with the liquid under conditions that permit binding of SEQ ID NO: 14 to the immobilized affinity ligand; and (d) eluting said ubiquitin mutein of SEQ NO: 14 or of muteins with at least 80% identity thereto from said affinity purification matrix, preferably with elution pH of 3.8, thereby obtaining an eluate containing said ubiquitin mutein with purity of at least 90%, preferably 100%, as determined via SDS-PAGE.

In some embodiments, the method of affinity purification of ubiquitin mutein of SEQ ID NO: 13, or of muteins with at least 80% identity thereto, is comprising: (a) providing a liquid that contains ubiquitin mutein of of SEQ ID NO: 13, or of muteins with at least 80% identity thereto; (b) providing an affinity separation matrix comprising a fusion protein comprising the affinity ligand of SEQ ID NOs: 1-5, 21 coupled to said affinity separation matrix; (c) contacting said affinity separation matrix with the liquid under conditions that permit binding of SEQ ID NO: 13, or of muteins with at least 80% identity thereto to the immobilized affinity ligand; and (d) eluting said ubiquitin mutein of SEQ ID NO: 13, or of muteins with at least 80% identity thereto matrix, preferably with elution pH of 4.3, thereby obtaining an eluate containing said ubiquitin mutein with purity of at least 90%, preferably 100%, as determined via SDS-PAGE.

In some embodiments, the method of affinity purification of ubiquitin mutein of SEQ ID NO: 10 or SEQ ID NO: 11 or SEQ ID NO: 17 is comprising: (a) providing a liquid that ubiquitin muteins of SEQ ID NO: 10 or SEQ ID NO: 11 or SEQ ID NO: 17, or of muteins with at least 80% identity thereto, respectively; (b) providing an affinity separation matrix comprising a fusion protein comprising the affinity ligand of SEQ ID NO: 1-5, 21 coupled to said affinity separation matrix; (c) contacting said affinity separation matrix with the liquid under conditions that permit binding of SEQ ID NO: 10 or SEQ ID NO: 11 or SEQ ID NO: 17 or of muteins with at least 80% identity thereto, respectively, to the immobilized affinity ligand; and (d) eluting said ubiquitin mutein of SEQ NO: 10 or SEQ ID NO: 11 or SEQ ID NO: 17 or of muteins with at least 80% identity thereto, respectively, from said affinity purification matrix, preferably with elution pH of 4.5, thereby obtaining an eluate containing said ubiquitin mutein with purity of 90%, as determined via SDS-PAGE.

Methods to determine the presence of ubiquitin mutein. Further, in some embodiments, the affinity ligand as described herein or the fusion protein as described herein are used in methods to determine the presence of a ubiquitin or a ubiquitin muteins. Some embodiments relate to a method of analyzing the presence of a ubiquitin or a ubiquitin mutein in liquid samples, the method comprising the following steps: (a) providing a liquid that contains ubiquitin or a ubiquitin mutein, (b) providing the affinity ligand, (c) contacting the liquid that contains ubiquitin or ubiquitin muteins with the affinity ligand as described herein under conditions that permit binding of the at least one affinity ligand as described herein, (d) eluting the ubiquitin or ubiquitin mutein, and optionally, (e) determining the amount of ubiquitin or the ubiquitin mutein in the liquid of (a). The elution pH may be 3.5 or higher.

Further embodiments relate to a method of quantification of a ubiquitin or a ubiquitin mutein, the method comprising: (a) providing a liquid that contains ubiquitin or ubiquitin muteins; (b) providing a matrix to which the affinity ligand as described herein has been covalently coupled; (c) contacting said affinity purification matrix with the liquid under conditions that permit binding of the at least one affinity ligand as described herein; (d) eluting said ubiquitin or ubiquitin muteins; and optionally, (e) quantitating the amount of eluted ubiquitin or ubiquitin muteins. Methods to determine the presence of ubiquitin muteins in liquid samples might be quantitative or qualitative. Such methods are well known to the skilled person and can be selected, for instance but limited to, from the following methods that are well established in the art: enzyme-linked immunosorbent assay (ELISA), enzymatic reactions, surface plasmon resonance (SPR), or chromatography.

Polynucleotides, vectors, host cells. One embodiment covers an isolated polynucleotide or nucleic acid molecule encoding an affinity ligand as disclosed herein. A further embodiment also encompasses proteins encoded by the polynucleotides as disclosed herein.

Further provided is a vector, in particular an expression vector, comprising the isolated polynucleotide or nucleic acid molecule of the invention, as well as a host cell comprising the isolated polynucleotide or the expression vector. For example, one or more polynucleotides, which encode a polypeptide as disclosed herein may be expressed in a suitable host and the produced protein can be isolated. A vector means any molecule or entity (e.g., nucleic acid, plasmid, bacteriophage or virus) that can be used for transfer of protein-encoding information into a host cell. Suitable vectors that may be applied in the present invention are known in the art.

Furthermore, an isolated cell comprising a polynucleotide or nucleic acid, or a vector as disclosed herein is provided. Suitable host cells include prokaryotes or eukaryotes, for example a bacterial host cell, a yeast host cell or a non-human host cell carrying a vector. Suitable bacterial expression host cells or systems are known in the art. Various mammalian or insect cell culture systems as known in the art can also be employed to express recombinant proteins.

Method of producing a protein of the invention. In a further embodiment, a method for the production of the affinity ligand as described is provided, the method comprising the step(s): (a) culturing a (suitable) host cell under conditions suitable for the expression of the affinity ligand so as to obtain said affinity ligand; and (b) optionally isolating said affinity ligand. Suitable conditions for culturing a prokaryotic or eukaryotic host are well known to a person skilled in the art.

The affinity ligand may be prepared by any conventional and well-known techniques such as plain organic synthetic strategies, solid phase-assisted synthesis techniques, or by commercially available automated synthesizers. They may also be prepared by conventional recombinant techniques, alone or in combination with conventional synthetic techniques.

In one embodiment, a method for the preparation of the affinity ligand is provided, as detailed above, said method comprising the steps: (a) providing a nucleic acid molecule encoding the binding polypeptide; (b) introducing said nucleic acid molecule into an expression vector; (c) introducing said expression vector into a host cell; (d) culturing the host cell in a culture medium; (e) subjecting the host cell to culturing conditions suitable for expression thereby producing a binding polypeptide; optionally (f) isolating the protein or polypeptide produced in step (e); and (g) optionally conjugating the protein or polypeptide to a solid matrix as described above. In various embodiments of the present invention the production of the affinity ligand is performed by cell-free in vitro transcription and translation.

The following Examples are provided for further illustration of the invention. The invention, however, is not limited thereto, and the following Examples merely show the practicability of the invention on the basis of the above description.

Aspects and embodiments of the invention include:

1a. An affinity ligand comprising an amino acid sequence with at least 90%, preferably 92%, more preferably 94%, even more preferably 96%, sequence identity to at least 56 amino acids of the amino acid sequence of SEQ ID NO: 1, wherein the affinity ligand is stable under alkaline conditions of 0.1 M NaOH incubation for at least 6 h, and wherein the affinity ligand has a binding affinity of less than 100 nM for a ubiquitin mutein,
and wherein the affinity ligand has one or more of:
(i) a methionine (M) at a position corresponding to position 4 of SEQ ID NO: 1,
(ii) an aspartate (D) at a position corresponding to position 5 of SEQ ID NO: 1,
(iii) a phenylalanine (F) at a position corresponding to position 7 of SEQ ID NO: 1,
(iv) a tyrosine (Y) at a position corresponding to position 10 of SEQ ID NO: 1,
(v) a tryptophan (W) at a position corresponding to position 13 of SEQ ID NO: 1,
(vi) an alanine (A) at a position corresponding to position 14 of SEQ ID NO: 1,
(vii) a glutamate (E) at a position corresponding to position 17 of SEQ ID NO: 1,
(viii) an alanine (A) at a position corresponding to position 18 of SEQ ID NO: 1,
(ix) a glutamate (E) at a position corresponding to position 24 of SEQ ID NO: 1,
(x) a methionine (M) at a position corresponding to position 25 of SEQ ID NO: 1,
(xi) a lysine (K) at a position corresponding to position 27 of SEQ ID NO: 1,
(xii) an aspartate (D) at a position corresponding to position 28 of SEQ ID NO: 1,
(xiii) a glutamine (Q) at a position corresponding to position 29 of SEQ ID NO: 1,
(xiv) a tryptophan (W) at a position corresponding to position 32 of SEQ ID NO: 1,
(xv) a methionine (M) at a position corresponding to position 35 of SEQ ID NO: 1, and/or
(xvi) a phenylalanine (F) at a position corresponding to position 36 of SEQ ID NO: 1.
   The affinity ligand may have two or three of:
(i) a methionine (M) at a position corresponding to position 4 of SEQ ID NO: 1,
(ii) an aspartate (D) at a position corresponding to position 5 of SEQ ID NO: 1,
(iii) a phenylalanine (F) at a position corresponding to position 7 of SEQ ID NO: 1,
(iv) a tyrosine (Y) at a position corresponding to position 10 of SEQ ID NO: 1,
(v) a tryptophan (W) at a position corresponding to position 13 of SEQ ID NO: 1,
(vi) an alanine (A) at a position corresponding to position 14 of SEQ ID NO: 1,
(vii) a glutamate (E) at a position corresponding to position 17 of SEQ ID NO: 1,
(viii) an alanine (A) at a position corresponding to position 18 of SEQ ID NO: 1,
(ix) a glutamate (E) at a position corresponding to position 24 of SEQ ID NO: 1,
(x) a methionine (M) at a position corresponding to position 25 of SEQ ID NO: 1,
(xi) a lysine (K) at a position corresponding to position 27 of SEQ ID NO: 1,
(xii) an aspartate (D) at a position corresponding to position 28 of SEQ ID NO: 1,
(xiii) a glutamine (Q) at a position corresponding to position 29 of SEQ ID NO: 1,
(xiv) a tryptophan (W) at a position corresponding to position 32 of SEQ ID NO: 1,
(xv) a methionine (M) at a position corresponding to position 35 of SEQ ID NO: 1, and/or
(xvi) a phenylalanine (F) at a position corresponding to position 36 of SEQ ID NO: 1.
   The affinity ligand may have four, five or six of:
(i) a methionine (M) at a position corresponding to position 4 of SEQ ID NO: 1,
(ii) an aspartate (D) at a position corresponding to position 5 of SEQ ID NO: 1,
(iii) a phenylalanine (F) at a position corresponding to position 7 of SEQ ID NO: 1,
(iv) a tyrosine (Y) at a position corresponding to position 10 of SEQ ID NO: 1,
(v) a tryptophan (W) at a position corresponding to position 13 of SEQ ID NO: 1,
(vi) an alanine (A) at a position corresponding to position 14 of SEQ ID NO: 1,
(vii) a glutamate (E) at a position corresponding to position 17 of SEQ ID NO: 1,
(viii) an alanine (A) at a position corresponding to position 18 of SEQ ID NO: 1,
(ix) a glutamate (E) at a position corresponding to position 24 of SEQ ID NO: 1,
(x) a methionine (M) at a position corresponding to position 25 of SEQ ID NO: 1,
(xi) a lysine (K) at a position corresponding to position 27 of SEQ ID NO: 1,
(xii) an aspartate (D) at a position corresponding to position 28 of SEQ ID NO: 1,
(xiii) a glutamine (Q) at a position corresponding to position 29 of SEQ ID NO: 1,
(xiv) a tryptophan (W) at a position corresponding to position 32 of SEQ ID NO: 1,
(xv) a methionine (M) at a position corresponding to position 35 of SEQ ID NO: 1, and/or
(xvi) a phenylalanine (F) at a position corresponding to position 36 of SEQ ID NO: 1.
   The affinity ligand may have seven, eight or nine of:
(i) a methionine (M) at a position corresponding to position 4 of SEQ ID NO: 1,
(ii) an aspartate (D) at a position corresponding to position 5 of SEQ ID NO: 1,
(iii) a phenylalanine (F) at a position corresponding to position 7 of SEQ ID NO: 1,
(iv) a tyrosine (Y) at a position corresponding to position 10 of SEQ ID NO: 1,
(v) a tryptophan (W) at a position corresponding to position 13 of SEQ ID NO: 1,
(vi) an alanine (A) at a position corresponding to position 14 of SEQ ID NO: 1,
(vii) a glutamate (E) at a position corresponding to position 17 of SEQ ID NO: 1,
(viii) an alanine (A) at a position corresponding to position 18 of SEQ ID NO: 1,
(ix) a glutamate (E) at a position corresponding to position 24 of SEQ ID NO: 1,
(x) a methionine (M) at a position corresponding to position 25 of SEQ ID NO: 1,
(xi) a lysine (K) at a position corresponding to position 27 of SEQ ID NO: 1,
(xii) an aspartate (D) at a position corresponding to position 28 of SEQ ID NO: 1,
(xiii) a glutamine (Q) at a position corresponding to position 29 of SEQ ID NO: 1,
(xiv) a tryptophan (W) at a position corresponding to position 32 of SEQ ID NO: 1,
(xv) a methionine (M) at a position corresponding to position 35 of SEQ ID NO: 1, and/or
(xvi) a phenylalanine (F) at a position corresponding to position 36 of SEQ ID NO: 1.
   The affinity ligand may have ten, eleven or twelve of:
(i) a methionine (M) at a position corresponding to position 4 of SEQ ID NO: 1,
(ii) an aspartate (D) at a position corresponding to position 5 of SEQ ID NO: 1,
(iii) a phenylalanine (F) at a position corresponding to position 7 of SEQ ID NO: 1,
(iv) a tyrosine (Y) at a position corresponding to position 10 of SEQ ID NO: 1,
(v) a tryptophan (W) at a position corresponding to position 13 of SEQ ID NO: 1,
(vi) an alanine (A) at a position corresponding to position 14 of SEQ ID NO: 1,
(vii) a glutamate (E) at a position corresponding to position 17 of SEQ ID NO: 1,
(viii) an alanine (A) at a position corresponding to position 18 of SEQ ID NO: 1,
(ix) a glutamate (E) at a position corresponding to position 24 of SEQ ID NO: 1,
(x) a methionine (M) at a position corresponding to position 25 of SEQ ID NO: 1,
(xi) a lysine (K) at a position corresponding to position 27 of SEQ ID NO: 1,
(xii) an aspartate (D) at a position corresponding to position 28 of SEQ ID NO: 1,
(xiii) a glutamine (Q) at a position corresponding to position 29 of SEQ ID NO: 1,
(xiv) a tryptophan (W) at a position corresponding to position 32 of SEQ ID NO: 1,
(xv) a methionine (M) at a position corresponding to position 35 of SEQ ID NO: 1, and/or
(xvi) a phenylalanine (F) at a position corresponding to position 36 of SEQ ID NO: 1.
   1b. The present invention encompasses an affinity ligand according to the above item 1a, comprising an amino acid sequence with at least 90%, preferably 92%, more preferably 94%, even more preferably 96%, sequence identity to at least 56 amino acids of the amino acid sequence of SEQ ID NO: 1, wherein the affinity ligand is stable under alkaline conditions of 0.25 M NaOH incubation for at least 6 h, and wherein the affinity ligand has a binding affinity of less than 10 nM, preferably less than 1 nM, for a ubiquitin mutein, wherein the affinity ligand comprises one or more of the following amino acid substitutions in the amino acid sequence of SEQ ID NO: 1: M4K, D5F, F7E, Y10Q, A18H, M25E, and/or K27R.
   2a. An affinity ligand comprising an amino acid sequence with at least 90%, preferably 92%, more preferably 94%, even more preferably 96%, sequence identity to at least 56 amino acids of the amino acid sequence of SEQ ID NO: 3, wherein the affinity ligand is stable under alkaline conditions of 0.1 M NaOH incubation for at least 6 h, and wherein the affinity ligand has a binding affinity of less than 100 nM for a ubiquitin mutein,
and wherein the affinity ligand has one or more of:
(i) a methionine (M) at a position corresponding to position 4 of SEQ ID NO: 3,
(ii) an aspartate (D) at a position corresponding to position 5 of SEQ ID NO: 3,
(iii) a phenylalanine (F) at a position corresponding to position 7 of SEQ ID NO: 3,
(iv) a tyrosine (Y) at a position corresponding to position 10 of SEQ ID NO: 3,
(v) a tryptophan (W) at a position corresponding to position 13 of SEQ ID NO: 3,
(vi) an alanine (A) at a position corresponding to position 14 of SEQ ID NO: 3,
(vii) a glutamate (E) at a position corresponding to position 17 of SEQ ID NO: 3,
(viii) an alanine (A) at a position corresponding to position 18 of SEQ ID NO: 3,
(ix) a glutamate (E) at a position corresponding to position 24 of SEQ ID NO: 3,
(x) a methionine (M) at a position corresponding to position 25 of SEQ ID NO: 3,
(xi) a lysine (K) at a position corresponding to position 27 of SEQ ID NO: 3,
(xii) an aspartate (D) at a position corresponding to position 28 of SEQ ID NO: 3,
(xiii) a glutamine (Q) at a position corresponding to position 29 of SEQ ID NO: 3,
(xiv) a tryptophan (W) at a position corresponding to position 32 of SEQ ID NO: 3,
(xv) a methionine (M) at a position corresponding to position 35 of SEQ ID NO: 3, and/or
(xvi) a phenylalanine (F) at a position corresponding to position 36 of SEQ ID NO: 3.
   2b. An affinity ligand comprising an amino acid sequence with at least 90%, preferably 92%, more preferably 94%, even more preferably 96%, sequence identity to at least 56 amino acids of the amino acid sequence of SEQ ID NO: 5, wherein the affinity ligand is stable under alkaline conditions of 0.1 M NaOH incubation for at least 6 h, and wherein the affinity ligand has a binding affinity of less than 100 nM for a ubiquitin mutein,
and wherein the affinity ligand has one or more of:
(i) a methionine (M) at a position corresponding to position 4 of SEQ ID NO: 5,
(ii) an aspartate (D) at a position corresponding to position 5 of SEQ ID NO: 5,
(iii) a phenylalanine (F) at a position corresponding to position 7 of SEQ ID NO: 5,
(iv) a tyrosine (Y) at a position corresponding to position 10 of SEQ ID NO: 5,
(v) a tryptophan (W) at a position corresponding to position 13 of SEQ ID NO: 5,
(vi) an alanine (A) at a position corresponding to position 14 of SEQ ID NO: 5,
(vii) a glutamate (E) at a position corresponding to position 17 of SEQ ID NO: 5,
(viii) an alanine (A) at a position corresponding to position 18 of SEQ ID NO: 5,
(ix) a glutamate (E) at a position corresponding to position 24 of SEQ ID NO: 5,
(x) a methionine (M) at a position corresponding to position 25 of SEQ ID NO: 5,
(xi) a lysine (K) at a position corresponding to position 27 of SEQ ID NO: 5,
(xii) an aspartate (D) at a position corresponding to position 28 of SEQ ID NO: 5,
(xiii) a glutamine (Q) at a position corresponding to position 29 of SEQ ID NO: 5,
(xiv) a tryptophan (W) at a position corresponding to position 32 of SEQ ID NO: 5,
(xv) a methionine (M) at a position corresponding to position 35 of SEQ ID NO: 5, and/or
(xvi) a phenylalanine (F) at a position corresponding to position 36 of SEQ ID NO: 5.
   2c. The present invention encompasses an affinity ligand according to the above item 2a or 2b, comprising an amino acid sequence with at least 90%, preferably 92%, more preferably 94%, even more preferably 96%, sequence identity to at least 56 amino acids of the amino acid sequence of SEQ ID NO: 3 or 5, wherein the affinity ligand is stable under alkaline conditions of 0.25 M NaOH incubation for at least 6 h, and wherein the affinity ligand has a binding affinity of less than 10 nM, preferably less than 1 nM, for a ubiquitin mutein, wherein the affinity ligand comprises one or more of the following amino acid substitutions in the amino acid sequence of SEQ ID NO: 3 or 5: M4K, D5F, F7E, Y10Q, A18H, M25E, and/or K27R.
   The affinity ligand may have two or three of:
(i) a methionine (M) at a position corresponding to position 4 of SEQ ID NO: 3 or 5,
(ii) an aspartate (D) at a position corresponding to position 5 of SEQ ID NO: 3 or 5,
(iii) a phenylalanine (F) at a position corresponding to position 7 of SEQ ID NO: 3 or 5,
(iv) a tyrosine (Y) at a position corresponding to position 10 of SEQ ID NO: 3 or 5,
(v) a tryptophan (W) at a position corresponding to position 13 of SEQ ID NO: 3 or 5,
(vi) an alanine (A) at a position corresponding to position 14 of SEQ ID NO: 3 or 5,
(vii) a glutamate (E) at a position corresponding to position 17 of SEQ ID NO: 3 or 5,
(viii) an alanine (A) at a position corresponding to position 18 of SEQ ID NO: 3 or 5,
(ix) a glutamate (E) at a position corresponding to position 24 of SEQ ID NO: 3 or 5,
(x) a methionine (M) at a position corresponding to position 25 of SEQ ID NO: 3 or 5,
(xi) a lysine (K) at a position corresponding to position 27 of SEQ ID NO: 3 or 5,
(xii) an aspartate (D) at a position corresponding to position 28 of SEQ ID NO: 3 or 5,
(xiii) a glutamine (Q) at a position corresponding to position 29 of SEQ ID NO: 3 or 5,
(xiv) a tryptophan (W) at a position corresponding to position 32 of SEQ ID NO: 3 or 5,
(xv) a methionine (M) at a position corresponding to position 35 of SEQ ID NO: 3 or 5, and/or
(xvi) a phenylalanine (F) at a position corresponding to position 36 of SEQ ID NO: 3 or 5.
   The affinity ligand may have four, five or six of:
(i) a methionine (M) at a position corresponding to position 4 of SEQ ID NO: 3 or 5,
(ii) an aspartate (D) at a position corresponding to position 5 of SEQ ID NO: 3 or 5,
(iii) a phenylalanine (F) at a position corresponding to position 7 of SEQ ID NO: 3 or 5,
(iv) a tyrosine (Y) at a position corresponding to position 10 of SEQ ID NO: 3 or 5,
(v) a tryptophan (W) at a position corresponding to position 13 of SEQ ID NO: 3 or 5,
(vi) an alanine (A) at a position corresponding to position 14 of SEQ ID NO: 3 or 5,
(vii) a glutamate (E) at a position corresponding to position 17 of SEQ ID NO: 3 or 5,
(viii) an alanine (A) at a position corresponding to position 18 of SEQ ID NO: 3 or 5,
(ix) a glutamate (E) at a position corresponding to position 24 of SEQ ID NO: 3 or 5,
(x) a methionine (M) at a position corresponding to position 25 of SEQ ID NO: 3 or 5,
(xi) a lysine (K) at a position corresponding to position 27 of SEQ ID NO: 3 or 5,
(xii) an aspartate (D) at a position corresponding to position 28 of SEQ ID NO: 3 or 5,
(xiii) a glutamine (Q) at a position corresponding to position 29 of SEQ ID NO: 3 or 5,
(xiv) a tryptophan (W) at a position corresponding to position 32 of SEQ ID NO: 3 or 5,
(xv) a methionine (M) at a position corresponding to position 35 of SEQ ID NO: 3 or 5, and/or
(xvi) a phenylalanine (F) at a position corresponding to position 36 of SEQ ID NO: 3 or 5.
   The affinity ligand may have seven, eight or nine of:
(i) a methionine (M) at a position corresponding to position 4 of SEQ ID NO: 3 or 5,
(ii) an aspartate (D) at a position corresponding to position 5 of SEQ ID NO: 3 or 5,
(iii) a phenylalanine (F) at a position corresponding to position 7 of SEQ ID NO: 3 or 5,
(iv) a tyrosine (Y) at a position corresponding to position 10 of SEQ ID NO: 3 or 5,
(v) a tryptophan (W) at a position corresponding to position 13 of SEQ ID NO: 3 or 5,
(vi) an alanine (A) at a position corresponding to position 14 of SEQ ID NO: 3 or 5,
(vii) a glutamate (E) at a position corresponding to position 17 of SEQ ID NO: 3 or 5,
(viii) an alanine (A) at a position corresponding to position 18 of SEQ ID NO: 3 or 5,
(ix) a glutamate (E) at a position corresponding to position 24 of SEQ ID NO: 3 or 5,
(x) a methionine (M) at a position corresponding to position 25 of SEQ ID NO: 3 or 5,
(xi) a lysine (K) at a position corresponding to position 27 of SEQ ID NO: 3 or 5,
(xii) an aspartate (D) at a position corresponding to position 28 of SEQ ID NO: 3 or 5,
(xiii) a glutamine (Q) at a position corresponding to position 29 of SEQ ID NO: 3 or 5,
(xiv) a tryptophan (W) at a position corresponding to position 32 of SEQ ID NO: 3 or 5,
(xv) a methionine (M) at a position corresponding to position 35 of SEQ ID NO: 3 or 5, and/or
(xvi) a phenylalanine (F) at a position corresponding to position 36 of SEQ ID NO: 3 or 5.
   The affinity ligand may have ten, eleven or twelve of:
(i) a methionine (M) at a position corresponding to position 4 of SEQ ID NO: 3 or 5,
(ii) an aspartate (D) at a position corresponding to position 5 of SEQ ID NO: 3 or 5,
(iii) a phenylalanine (F) at a position corresponding to position 7 of SEQ ID NO: 3 or 5,
(iv) a tyrosine (Y) at a position corresponding to position 10 of SEQ ID NO: 3 or 5,
(v) a tryptophan (W) at a position corresponding to position 13 of SEQ ID NO: 3 or 5,
(vi) an alanine (A) at a position corresponding to position 14 of SEQ ID NO: 3 or 5,
(vii) a glutamate (E) at a position corresponding to position 17 of SEQ ID NO: 3 or 5,
(viii) an alanine (A) at a position corresponding to position 18 of SEQ ID NO: 3 or 5,
(ix) a glutamate (E) at a position corresponding to position 24 of SEQ ID NO: 3 or 5,
(x) a methionine (M) at a position corresponding to position 25 of SEQ ID NO: 3 or 5,
(xi) a lysine (K) at a position corresponding to position 27 of SEQ ID NO: 3 or 5,
(xii) an aspartate (D) at a position corresponding to position 28 of SEQ ID NO: 3 or 5,
(xiii) a glutamine (Q) at a position corresponding to position 29 of SEQ ID NO: 3 or 5,
(xiv) a tryptophan (W) at a position corresponding to position 32 of SEQ ID NO: 3 or 5,
(xv) a methionine (M) at a position corresponding to position 35 of SEQ ID NO: 3 or 5, and/or
(xvi) a phenylalanine (F) at a position corresponding to position 36 of SEQ ID NO: 3 or 5.
   The embodiments described elsewhere herein in regard to the X positions in SEQ ID NO: 5 fully apply to, i.e., can be combined with, the above-described aspect of the invention.
   3. An affinity ligand comprising an amino acid sequence with at least 90%, preferably 92%, more preferably 94%, even more preferably 96%, sequence identity to at least 56 amino acids of the amino acid sequence of SEQ ID NO: 21, wherein the affinity ligand is stable under alkaline conditions of 0.1 M NaOH incubation for at least 6 h, and wherein the affinity ligand has a binding affinity of less than 100 nM for a ubiquitin mutein,
and wherein the affinity ligand has one or more of:
(i) an arginine (R) at a position corresponding to position 4 of SEQ ID NO: 21,
(ii) a serine (S) at a position corresponding to position 5 of SEQ ID NO: 21,
(iii) a glutamate (E) at a position corresponding to position 7 of SEQ ID NO: 21,
(iv) a tyrosine (Y) at a position corresponding to position 10 of SEQ ID NO: 21,
(v) a tryptophan (W) at a position corresponding to position 13 of SEQ ID NO: 21,
(vi) an alanine (A) at a position corresponding to position 14 of SEQ ID NO: 21,
(vii) a glutamate (E) at a position corresponding to position 17 of SEQ ID NO: 21,
(viii) an alanine (A) at a position corresponding to position 18 of SEQ ID NO: 21,
(ix) a glutamate (E) at a position corresponding to position 24 of SEQ ID NO: 21,
(x) a methionine (M) at a position corresponding to position 25 of SEQ ID NO: 21,
(xi) a lysine (K) at a position corresponding to position 27 of SEQ ID NO: 21,
(xii) an aspartate (D) at a position corresponding to position 28 of SEQ ID NO: 21,
(xiii) a glutamine (Q) at a position corresponding to position 29 of SEQ ID NO: 21,
(xiv) a tryptophan (W) at a position corresponding to position 32 of SEQ ID NO: 21,
(xv) a methionine (M) at a position corresponding to position 35 of SEQ ID NO: 21, and/or
(xvi) a phenylalanine (F) at a position corresponding to position 36 of SEQ ID NO: 21.
   The affinity ligand may have two or three of:
(i) an arginine (R) at a position corresponding to position 4 of SEQ ID NO: 21,
(ii) a serine (S) at a position corresponding to position 5 of SEQ ID NO: 21,
(iii) a glutamate (E) at a position corresponding to position 7 of SEQ ID NO: 21,
(iv) a tyrosine (Y) at a position corresponding to position 10 of SEQ ID NO: 21,
(v) a tryptophan (W) at a position corresponding to position 13 of SEQ ID NO: 21,
(vi) an alanine (A) at a position corresponding to position 14 of SEQ ID NO: 21,
(vii) a glutamate (E) at a position corresponding to position 17 of SEQ ID NO: 21,
(viii) an alanine (A) at a position corresponding to position 18 of SEQ ID NO: 21,
(ix) a glutamate (E) at a position corresponding to position 24 of SEQ ID NO: 21,
(x) a methionine (M) at a position corresponding to position 25 of SEQ ID NO: 21,
(xi) a lysine (K) at a position corresponding to position 27 of SEQ ID NO: 21,
(xii) an aspartate (D) at a position corresponding to position 28 of SEQ ID NO: 21,
(xiii) a glutamine (Q) at a position corresponding to position 29 of SEQ ID NO: 21,
(xiv) a tryptophan (W) at a position corresponding to position 32 of SEQ ID NO: 21,
(xv) a methionine (M) at a position corresponding to position 35 of SEQ ID NO: 21, and/or
(xvi) a phenylalanine (F) at a position corresponding to position 36 of SEQ ID NO: 21.
   The affinity ligand may have four, five or six of:
(i) an arginine (R) at a position corresponding to position 4 of SEQ ID NO: 21,
(ii) a serine (S) at a position corresponding to position 5 of SEQ ID NO: 21,
(iii) a glutamate (E) at a position corresponding to position 7 of SEQ ID NO: 21,
(iv) a tyrosine (Y) at a position corresponding to position 10 of SEQ ID NO: 21,
(v) a tryptophan (W) at a position corresponding to position 13 of SEQ ID NO: 21,
(vi) an alanine (A) at a position corresponding to position 14 of SEQ ID NO: 21,
(vii) a glutamate (E) at a position corresponding to position 17 of SEQ ID NO: 21,
(viii) an alanine (A) at a position corresponding to position 18 of SEQ ID NO: 21,
(ix) a glutamate (E) at a position corresponding to position 24 of SEQ ID NO: 21,
(x) a methionine (M) at a position corresponding to position 25 of SEQ ID NO: 21,
(xi) a lysine (K) at a position corresponding to position 27 of SEQ ID NO: 21,
(xii) an aspartate (D) at a position corresponding to position 28 of SEQ ID NO: 21,
(xiii) a glutamine (Q) at a position corresponding to position 29 of SEQ ID NO: 21,
(xiv) a tryptophan (W) at a position corresponding to position 32 of SEQ ID NO: 21,
(xv) a methionine (M) at a position corresponding to position 35 of SEQ ID NO: 21, and/or
(xvi) a phenylalanine (F) at a position corresponding to position 36 of SEQ ID NO: 21.
   The affinity ligand may have seven, eight or nine of:
(i) an arginine (R) at a position corresponding to position 4 of SEQ ID NO: 21,
(ii) a serine (S) at a position corresponding to position 5 of SEQ ID NO: 21,
(iii) a glutamate (E) at a position corresponding to position 7 of SEQ ID NO: 21,
(iv) a tyrosine (Y) at a position corresponding to position 10 of SEQ ID NO: 21,
(v) a tryptophan (W) at a position corresponding to position 13 of SEQ ID NO: 21,
(vi) an alanine (A) at a position corresponding to position 14 of SEQ ID NO: 21,
(vii) a glutamate (E) at a position corresponding to position 17 of SEQ ID NO: 21,
(viii) an alanine (A) at a position corresponding to position 18 of SEQ ID NO: 21,
(ix) a glutamate (E) at a position corresponding to position 24 of SEQ ID NO: 21,
(x) a methionine (M) at a position corresponding to position 25 of SEQ ID NO: 21,
(xi) a lysine (K) at a position corresponding to position 27 of SEQ ID NO: 21,
(xii) an aspartate (D) at a position corresponding to position 28 of SEQ ID NO: 21,
(xiii) a glutamine (Q) at a position corresponding to position 29 of SEQ ID NO: 21,
(xiv) a tryptophan (W) at a position corresponding to position 32 of SEQ ID NO: 21,
(xv) a methionine (M) at a position corresponding to position 35 of SEQ ID NO: 21, and/or
(xvi) a phenylalanine (F) at a position corresponding to position 36 of SEQ ID NO: 21.
   The affinity ligand may have ten, eleven or twelve of:
(i) an arginine (R) at a position corresponding to position 4 of SEQ ID NO: 21,
(ii) a serine (S) at a position corresponding to position 5 of SEQ ID NO: 21,
(iii) a glutamate (E) at a position corresponding to position 7 of SEQ ID NO: 21,
(iv) a tyrosine (Y) at a position corresponding to position 10 of SEQ ID NO: 21,
(v) a tryptophan (W) at a position corresponding to position 13 of SEQ ID NO: 21,
(vi) an alanine (A) at a position corresponding to position 14 of SEQ ID NO: 21,
(vii) a glutamate (E) at a position corresponding to position 17 of SEQ ID NO: 21,
(viii) an alanine (A) at a position corresponding to position 18 of SEQ ID NO: 21,
(ix) a glutamate (E) at a position corresponding to position 24 of SEQ ID NO: 21,
(x) a methionine (M) at a position corresponding to position 25 of SEQ ID NO: 21,
(xi) a lysine (K) at a position corresponding to position 27 of SEQ ID NO: 21,
(xii) an aspartate (D) at a position corresponding to position 28 of SEQ ID NO: 21,
(xiii) a glutamine (Q) at a position corresponding to position 29 of SEQ ID NO: 21,
(xiv) a tryptophan (W) at a position corresponding to position 32 of SEQ ID NO: 21,
(xv) a methionine (M) at a position corresponding to position 35 of SEQ ID NO: 21, and/or
(xvi) a phenylalanine (F) at a position corresponding to position 36 of SEQ ID NO: 21.
   4a. An affinity ligand comprising an amino acid sequence with at least 90%, preferably 92%, more preferably 94%, even more preferably 96%, sequence identity to the amino acid sequence of SEQ ID NO: 2, wherein the affinity ligand is stable under alkaline conditions of 0.1 M NaOH incubation for at least 6 h, and wherein the affinity ligand has a binding affinity of less than 100 nM for a ubiquitin mutein,
and wherein the affinity ligand has one or more of:
(i) a methionine (M) at a position corresponding to position 2 of SEQ ID NO: 2,
(ii) an aspartate (D) at a position corresponding to position 3 of SEQ ID NO: 2,
(iii) a phenylalanine (F) at a position corresponding to position 5 of SEQ ID NO: 2,
(iv) a tyrosine (Y) at a position corresponding to position 8 of SEQ ID NO: 2,
(v) a tryptophan (W) at a position corresponding to position 11 of SEQ ID NO: 2,
(vi) an alanine (A) at a position corresponding to position 12 of SEQ ID NO: 2,
(vii) a glutamate (E) at a position corresponding to position 15 of SEQ ID NO: 2,
(viii) an alanine (A) at a position corresponding to position 16 of SEQ ID NO: 2,
(ix) a glutamate (E) at a position corresponding to position 22 of SEQ ID NO: 2,
(x) a methionine (M) at a position corresponding to position 23 of SEQ ID NO: 2,
(xi) a lysine (K) at a position corresponding to position 25 of SEQ ID NO: 2,
(xii) an aspartate (D) at a position corresponding to position 26 of SEQ ID NO: 2,
(xiii) a glutamine (Q) at a position corresponding to position 27 of SEQ ID NO: 2,
(xiv) a tryptophan (W) at a position corresponding to position 30 of SEQ ID NO: 2,
(xv) a methionine (M) at a position corresponding to position 33 of SEQ ID NO: 2, and/or
(xvi) a phenylalanine (F) at a position corresponding to position 34 of SEQ ID NO: 2.
   4b. The present invention encompasses an affinity ligand according to the above item 4a, comprising an amino acid sequence with at least 90%, preferably 92%, more preferably 94%, even more preferably 96%, sequence identity to the amino acid sequence of SEQ ID NO: 2, wherein the affinity ligand is stable under alkaline conditions of 0.25 M NaOH incubation for at least 6 h, and wherein the affinity ligand has a binding affinity of less than 10 nM, preferably less than 1 nM, for a ubiquitin mutein, wherein the affinity ligand comprises one or more of the following amino acid substitutions in the amino acid sequence of SEQ ID NO: 2: M2K, D3F, F5E, Y8Q, A16H, M23E, and/or K25R.
   4c. An affinity ligand comprising an amino acid sequence with at least 90%, preferably 92%, more preferably 94%, even more preferably 96%, sequence identity to the amino acid sequence of SEQ ID NO: 4, wherein the affinity ligand is stable under alkaline conditions of 0.1 M NaOH incubation for at least 6 h, and wherein the affinity ligand has a binding affinity of less than 100 nM for a ubiquitin mutein,
and wherein the affinity ligand has one or more of:
(i) a methionine (M) at a position corresponding to position 2 of SEQ ID NO: 4,
(ii) an aspartate (D) at a position corresponding to position 3 of SEQ ID NO: 4,
(iii) a phenylalanine (F) at a position corresponding to position 5 of SEQ ID NO: 4,
(iv) a tyrosine (Y) at a position corresponding to position 8 of SEQ ID NO: 4,
(v) a tryptophan (W) at a position corresponding to position 11 of SEQ ID NO: 4,
(vi) an alanine (A) at a position corresponding to position 12 of SEQ ID NO: 4,
(vii) a glutamate (E) at a position corresponding to position 15 of SEQ ID NO: 4,
(viii) an alanine (A) at a position corresponding to position 16 of SEQ ID NO: 4,
(ix) a glutamate (E) at a position corresponding to position 22 of SEQ ID NO: 4,
(x) a methionine (M) at a position corresponding to position 23 of SEQ ID NO: 4,
(xi) a lysine (K) at a position corresponding to position 25 of SEQ ID NO: 4,
(xii) an aspartate (D) at a position corresponding to position 26 of SEQ ID NO: 4,
(xiii) a glutamine (Q) at a position corresponding to position 27 of SEQ ID NO: 4,
(xiv) a tryptophan (W) at a position corresponding to position 30 of SEQ ID NO: 4,
(xv) a methionine (M) at a position corresponding to position 33 of SEQ ID NO: 4, and/or
(xvi) a phenylalanine (F) at a position corresponding to position 34 of SEQ ID NO: 4. The affinity ligand may have two or three of:
(i) a methionine (M) at a position corresponding to position 2 of SEQ ID NO: 2 or 4,
(ii) an aspartate (D) at a position corresponding to position 3 of SEQ ID NO: 2 or 4,
(iii) a phenylalanine (F) at a position corresponding to position 5 of SEQ ID NO: 2 or 4,
(iv) a tyrosine (Y) at a position corresponding to position 8 of SEQ ID NO: 2 or 4,
(v) a tryptophan (W) at a position corresponding to position 11 of SEQ ID NO: 2 or 4,
(vi) an alanine (A) at a position corresponding to position 12 of SEQ ID NO: 2 or 4,
(vii) a glutamate (E) at a position corresponding to position 15 of SEQ ID NO: 2 or 4,
(viii) an alanine (A) at a position corresponding to position 16 of SEQ ID NO: 2 or 4,
(ix) a glutamate (E) at a position corresponding to position 22 of SEQ ID NO: 2 or 4,
(x) a methionine (M) at a position corresponding to position 23 of SEQ ID NO: 2 or 4,
(xi) a lysine (K) at a position corresponding to position 25 of SEQ ID NO: 2 or 4,
(xii) an aspartate (D) at a position corresponding to position 26 of SEQ ID NO: 2 or 4,
(xiii) a glutamine (Q) at a position corresponding to position 27 of SEQ ID NO: 2 or 4,
(xiv) a tryptophan (W) at a position corresponding to position 30 of SEQ ID NO: 2 or 4,
(xv) a methionine (M) at a position corresponding to position 33 of SEQ ID NO: 2 or 4, and/or
(xvi) a phenylalanine (F) at a position corresponding to position 34 of SEQ ID NO: 2 or 4.
   The affinity ligand may have four, five or six of:
(i) a methionine (M) at a position corresponding to position 2 of SEQ ID NO: 2 or 4,
(ii) an aspartate (D) at a position corresponding to position 3 of SEQ ID NO: 2 or 4,
(iii) a phenylalanine (F) at a position corresponding to position 5 of SEQ ID NO: 2 or 4,
(iv) a tyrosine (Y) at a position corresponding to position 8 of SEQ ID NO: 2 or 4,
(v) a tryptophan (W) at a position corresponding to position 11 of SEQ ID NO: 2 or 4,
(vi) an alanine (A) at a position corresponding to position 12 of SEQ ID NO: 2 or 4,
(vii) a glutamate (E) at a position corresponding to position 15 of SEQ ID NO: 2 or 4,
(viii) an alanine (A) at a position corresponding to position 16 of SEQ ID NO: 2 or 4,
(ix) a glutamate (E) at a position corresponding to position 22 of SEQ ID NO: 2 or 4,
(x) a methionine (M) at a position corresponding to position 23 of SEQ ID NO: 2 or 4,
(xi) a lysine (K) at a position corresponding to position 25 of SEQ ID NO: 2 or 4,
(xii) an aspartate (D) at a position corresponding to position 26 of SEQ ID NO: 2 or 4,
(xiii) a glutamine (Q) at a position corresponding to position 27 of SEQ ID NO: 2 or 4,
(xiv) a tryptophan (W) at a position corresponding to position 30 of SEQ ID NO: 2 or 4,
(xv) a methionine (M) at a position corresponding to position 33 of SEQ ID NO: 2 or 4, and/or
(xvi) a phenylalanine (F) at a position corresponding to position 34 of SEQ ID NO: 2 or 4.
   The affinity ligand may have seven, eight or nine of:
(i) a methionine (M) at a position corresponding to position 2 of SEQ ID NO: 2 or 4,
(ii) an aspartate (D) at a position corresponding to position 3 of SEQ ID NO: 2 or 4,
(iii) a phenylalanine (F) at a position corresponding to position 5 of SEQ ID NO: 2 or 4,
(iv) a tyrosine (Y) at a position corresponding to position 8 of SEQ ID NO: 2 or 4,
(v) a tryptophan (W) at a position corresponding to position 11 of SEQ ID NO: 2 or 4,
(vi) an alanine (A) at a position corresponding to position 12 of SEQ ID NO: 2 or 4,
(vii) a glutamate (E) at a position corresponding to position 15 of SEQ ID NO: 2 or 4,
(viii) an alanine (A) at a position corresponding to position 16 of SEQ ID NO: 2 or 4,
(ix) a glutamate (E) at a position corresponding to position 22 of SEQ ID NO: 2 or 4,
(x) a methionine (M) at a position corresponding to position 23 of SEQ ID NO: 2 or 4,
(xi) a lysine (K) at a position corresponding to position 25 of SEQ ID NO: 2 or 4,
(xii) an aspartate (D) at a position corresponding to position 26 of SEQ ID NO: 2 or 4,
(xiii) a glutamine (Q) at a position corresponding to position 27 of SEQ ID NO: 2 or 4,
(xiv) a tryptophan (W) at a position corresponding to position 30 of SEQ ID NO: 2 or 4,
(xv) a methionine (M) at a position corresponding to position 33 of SEQ ID NO: 2 or 4, and/or
(xvi) a phenylalanine (F) at a position corresponding to position 34 of SEQ ID NO: 2 or 4.
   The affinity ligand may have ten, eleven or twelve of:
(i) a methionine (M) at a position corresponding to position 2 of SEQ ID NO: 2 or 4,
(ii) an aspartate (D) at a position corresponding to position 3 of SEQ ID NO: 2 or 4,
(iii) a phenylalanine (F) at a position corresponding to position 5 of SEQ ID NO: 2 or 4,
(iv) a tyrosine (Y) at a position corresponding to position 8 of SEQ ID NO: 2 or 4,
(v) a tryptophan (W) at a position corresponding to position 11 of SEQ ID NO: 2 or 4,
(vi) an alanine (A) at a position corresponding to position 12 of SEQ ID NO: 2 or 4,
(vii) a glutamate (E) at a position corresponding to position 15 of SEQ ID NO: 2 or 4,
(viii) an alanine (A) at a position corresponding to position 16 of SEQ ID NO: 2 or 4,
(ix) a glutamate (E) at a position corresponding to position 22 of SEQ ID NO: 2 or 4,
(x) a methionine (M) at a position corresponding to position 23 of SEQ ID NO: 2 or 4,
(xi) a lysine (K) at a position corresponding to position 25 of SEQ ID NO: 2 or 4,
(xii) an aspartate (D) at a position corresponding to position 26 of SEQ ID NO: 2 or 4,
(xiii) a glutamine (Q) at a position corresponding to position 27 of SEQ ID NO: 2 or 4,
(xiv) a tryptophan (W) at a position corresponding to position 30 of SEQ ID NO: 2 or 4,
(xv) a methionine (M) at a position corresponding to position 33 of SEQ ID NO: 2 or 4, and/or
(xvi) a phenylalanine (F) at a position corresponding to position 34 of SEQ ID NO: 2 or 4.

All of the embodiments described elsewhere herein fully apply to, i.e., can be combined with, the above-described aspect of the invention.

### EXAMPLES

The following Examples are provided for further illustration of the invention. The invention, however, is not limited thereto, and the following Examples merely show the practicability of the invention on the basis of the above description. For a complete disclosure of the invention reference is made also to the literature cited in the application which is incorporated completely into the application by reference.

### Example 1. Selection and Screening of Affinity Ligand

Libraries. Proprietary cDNA libraries based on stable Protein A like variants (artificial mosaic proteins composed of fragments of Protein A domains and additional mutations) were synthesized by triplet technology (ThermoFisher Scientific—GeneArt, Germany) to achieve a well-balanced amino acid distribution with simultaneously exclusion of cysteine and other amino acid residues at randomized positions. The corresponding cDNA library was amplified in house by PCR and ligated into a pCD33-OmpA phagemid. Aliquots of the ligation mixture were used for electroporation of E. coli SS320 (Lucigen) to produce and purify the phage library to store them as cryo-stocks. Unless otherwise indicated, established recombinant genetic methods were used.

Selection by phage display: naive libraries were enriched against recombinant human ubiquitin mutein (ubiquitin mutein with 10 differences to ubiquitin; 86.8% identity to ubiquitin, monomer) as ON-target using phage display as selection system. In each round a pre-selection step was performed using empty Sigmablocker-blocked magnetic beads. The SIS-method was applied, which means that the affilin-carrying phages were incubated with the ON-target protein in solution and were captured afterwards for elution using either magnetic Epoxy M-270 Dynabeads or magnetic Pierce NHS-beads. E. coli SS320(Lucigene) were used for infection with cryo phage libraries and for reamplification of phage pools after each round. Amplification and purification of the phages were carried out using standard methods known to a skilled person. All four selection rounds were performed with the automated KingFisher-System (Thermo Fisher) to isolate and capture the desired phage-target complexes. Bound phages were eluted by trypsin and reamplified. The success of the selection was analyzed by phage-pool-ELISA in medium binding microtiter plate (Greiner Bio-One) coated with various ubiquitin muteins (125 ng/well), BSA (250 ng/well) or Sigmablocker. Bound phages were detected using α-M13 HRP conjugated antibody (GE Healthcare).

Results: Various phage display selection pools resulted in specific signals for the respective ON-target ubiquitin muteins. Controls with BSA or Sigmablocker showed for almost all pools no binding. Selected pools were sequenced, subcloned and proceed to high throughput screening. Enriched variants were selected as direct transfer for lab-scale production and purification.

Primary screening: Selection pools were proceeded to high throughput primary screening vs. ubiquitin muteins. Therefore ubiquitin muteins [c=2.5 μg/ml] was immobilized on a 384-well high binding plate and bound variants were detected by using Strep-Tactin labeled with horseradish peroxidase (absorption at 450/600 nm).

Secondary screening: Variants were selected for confirmation screening vs. ON-target: ubiquitin mutein [c=2.5 μg/ml] and OFF-target: BSA [c=2.5 μg/ml]. Hit criteria: signal of sample 10-fold larger than signal of OFF-target.

Tertiary screening: Variants with signal of sample 10-fold larger than signal of OFF-target were defined as hit for screening of low pH stability. Therefore cells were lysed at pH 3.0 with citrate buffer and neutralized with TRIS buffer. Soluble fractions were used for screening vs. ON-target: ubiquitin muteins [c=2.5 μg/ml] and OFF-target: BSA [c=2.5 μg/ml]. Hit criteria: signal of variants>0.7 and 8-fold larger than signal of OFF-target.

μ-scale purification: Variants defined as hit were sequenced, μ-scale purified (PhyNexus) and further analyzed.

Cloning of target binding phage pools into an expression vector. Selection pools showing specific binding to recombinant ubiquitin muteins in phage pool ELISA were amplified by PCR according to methods known in the art, cut with appropriate restriction nucleases and ligated into a derivative of the expression vector pET-28a (Merck, Germany).

### Site Directed Mutatgenesis:

The sequence of affinity ligand 187029 is shown in SEQ ID NO: 1. To generate an engineered ligand as shown in SEQ ID NO: 3, amino acids positions 8, 11, 42, 44, 46, 49, and 53 in SEQ ID NO: 1 were substituted by site directed mutagenesis: A8D, S11A, K42L, V441, G46A, Q49K, and S53A.

SEQ ID NO: 1 and SEQ NO: 3 have 90.8% amino acid identity.

SEQ ID NO: 2 is identical to SEQ ID NO: 1 in 56 amino acids. However, the two N-terminal amino acids are deleted in SEQ ID NO: 2.

SEQ ID NO: 4 is identical to SEQ ID NO: 3 in 56 amino acids. However, the two N-terminal amino acids are deleted in SEQ ID NO: 4.

The sequence of affinity ligand 187028 is shown in SEQ ID NO: 21.

### Example 2. Expression and Purification of Affinity Ligand and of Fusion Proteins Comprising the Affinity Ligand

Fusion proteins 214410, 214411, 214412 were expressed in an E. coli BL21(DE3) fermentation process using a pNP-017 vector system under regulation of a T7 promoter. Seed cultures were grown in preculture medium (34.5 g/L yeast extract, 0.61 g/L MgSO₄, 14.2 g/L K₂HPO₄, 0.5 g/L NH₄Cl, 50 μg/mL kanamycin). The fermentation process was performed in a bench-top bioreactor as a fed-batch process. The culture medium (17.25 g/L yeast extract, 0.61 g/L MgSO₄, 14.2 g/L K₂HPO₄, 0.5 g/L NH₄Cl, 50 μg/mL kanamycin) was inoculated with seed culture and the culture grown until the substrate was depleted (37° C., pH 7.1, 30% pO₂ saturation, aeration 2 VVM). Exponential feeding was performed with glucose as the main substrate (200 g/L glucose, 276 g/L yeast extract, 1.1 g/L MgSO4, 50 μg/mL kanamycin). Protein expression was induced by isopropyl β-D-1-thiogalactopyranoside (IPTG, end concentration of 1 mM) at 30° C. for 5 h at a constant feeding rate. To collect biomass cells were centrifuged at 12,000×g for 30 min. Bacterial pellets were stored at −20° C. before processing. Expression was analyzed via SDS-PAGE.

187029 was expressed in E. coli BL21(DE3) using a pET28a-based vector system in a bench-top bioreactor with autoinduction medium ZYM-5052. The main culture (0.5% glycerol, 0.2% lactose, 0.05% glucose, 0.5% yeast extract, 1.0% casamino acids, 25 mM Na₂HPO₄, 25 mM KH₂PO₄, 5 mM Na₂SO₄, 2 mM MgSO₄ and trace elements) was inoculated with a preculture grown in 2xYT (50 μg/ml kanamycin) and grown in a bench-top bioreactor (37° C., 30% pO2 saturation, aeration 2 VVM). Recombinant protein expression was induced by metabolizing glucose and subsequently allowing lactose to enter the cells. Cells were grown for approx. 6 hours. The harvest was performed as described in the previous process. Tagged proteins were purified by affinity and size exclusion chromatography. The initial capturing step was performed using StrepTactin Affinity resin (StrepTactin Superflow 5 ml, IBA, binding buffer: 100 mM TRIS, 150 mM NaCl, 1 mM EDTA, 1 mM DTT, pH 8.0; elution buffer: 100 mM TRIS, 150 mM NaCl, 1 mM EDTA, 1 mM DTT, 2.5 mM D-Desthiobiotin, pH 8.0) followed by a size exclusion chromatography (HiLoad Superdex 75 16/60 120 ml, GE Healthcare) in 20 mM Citric acid, 150 mM NaCl, 1 mM EDTA, 1 mM DTT pH 6.0 using an ÄKTA xpress system. Finally, 5 mM TCEP was added to the protein batch. For untagged proteins the capturing step was performed using hydrophobic interaction chromatography (Phenyl Sepharose HP, Cytiva, binding buffer: 20 mM BisTris, 1 mM EDTA, 1 M (NH₄)₂SO4 pH 7.0, elution buffer: 20 mM BisTris, 1 mM EDTA pH 7.0, gradient elution). Followed by an anion exchange chromatography step (Q Sepharose FF, Cytiva, binding buffer: 20 mM BisTris, 1 mM EDTA PH 7.0, elution buffer: 20 mM BisTris, 1 mM EDTA, 1 M NaCl pH 7.0, gradient elution). Polishing was performed by size exclusion chromatography using a Sephacryl S200 HR column (Cytiva; buffer: 20 mM citric acid, 150 mM NaCl, 1 mM EDTA, pH 6) using an ÄKTA avant system (Cytiva). Following SDS-PAGE analysis positive fractions were pooled and the protein concentrations were determined by absorbance measurement at 280 nm using the molar absorbent coefficient. Further analysis included RP-HPLC and SE-HPLC. Reversed phase chromatography (RP-HPLC) has been performed using a Dionex HPLC system and a PLRP-S (5 μm, 300 Å) column (Agilent). Analytic size exclusion chromatography (SE-HPLC) has been performed using a Dionex HPLC system and a Superdex75 increase 5/150 GL (GE Healthcare).

Fusion protein 214410 comprises SEQ ID NO: 1 (187029), fusion protein 214411 comprises SEQ ID NO: 2 (=SEQ ID NO: 1 del2N), and fusion protein 214412 comprises SEQ ID NO: 3. Fusion protein 220075 comprises SEQ ID NO: 2 and SEQ ID NO: 1 (187029). All affinity ligands are fused to three non-Ig binding proteins (SEQ ID NO. 9); the affinity ligand is located N-terminally of the fusion protein.

TABLE 1
SPR affinity ligands vs ubiquitin mutein (Affilin ®)-77404

| | | Comprises | | |
|---|---|---|---|---|
| SEQ ID | | SEQ ID | | SPR K_{D} |
| NO: | CID | NO: | se-HPLC | vs. Affilin ®-77404 |
| 1 | 187029 | 1 | 99.2% | <0.5 nM |
| 6 | 214410 | 1 | 99.6% | <0.5 nM |
| 7 | 214411 | 2 | 100% | <0.5 nM |
| 8 | 214412 | 3 | 100% | <0.5 nM |
| 22 | 220075 | 2 + 1 | 100% | <0.5 nM |

### Example 3. Binding Analysis by SPR

Purified proteins were immobilized via C-terminal Cysteine on a CM-5 sensor chip (GE Healthcare) using NHS/EDC after PDEA activation with a Biacore 3000 system (GE Healthcare). The chip was equilibrated with SPR running buffer (PBS 0.05% Tween pH 7.3). Soluble ubiquitin muteins (Affilin® proteins) applied to the chip in serial dilutions (different concentrations) with a flow rate of 30 μl/min. The association was performed for 120 seconds and the dissociation for 120 seconds. After each run, the chip surface was regenerated with 30 μl regeneration buffer (10 mM glycine pH 2.0) and equilibrated with running buffer. Affilin® proteins accumulated on the surface increasing the refractive index. This change in the refractive index was measured in real time and plotted as response or resonance units versus time.

Binding studies were carried out using the BIAcore 3000 (GE Healthcare); data evaluation was operated via the BIAevaluation 3.0 software, provided by the manufacturer, by the use of the Langmuir 1:1 model (RI=0). Evaluated dissociation constants (K_{D}) were standardized against the immobilized protein and indicated. Shown is the change in refractive index measured in real time and plotted as response or resonance unit [RU] versus time [sec] (see Table 2).

TABLE 2
SPR affinity of different ubiquitin muteins vs affinity ligands
of SEQ ID NO: 1 (187029) and SEQ ID NO: 21 (187028)

| Ubiquitin | Ubiquitin | SPR K_{D} | SPR K_{D} |
|---|---|---|---|
| mutein | mutein | vs. 187029 | vs. 187028 |
| 211883 | monomer | 52.9 nM | 66.2 nM |
| 139819 | monomer | 75.5 nM | 47.3 nM |
| 200053 | dimer | 5.9 nM | 2.5 nM |
| 77404 | dimer | 1.3 nM | 0.6 nM |

TABLE 2
SPR affinity of different ubiquitin muteins vs
multimers of SEQ ID NO: 1 and SEQ ID NO: 21

| | SPR K_{D} vs. | SPR K_{D} vs. | SPR K_{D} vs. | SPR K_{D} vs. | SPR K_{D} vs. | SPR K_{D} vs. |
|---|---|---|---|---|---|---|
| | SEQ21 | SEQ21 | SEQ 21 | SEQ1 | SEQ1 | SEQ1 |
| Ubiquitin | dimer | trimer | tetramer | dimer | trimer | tetramer |
| mutein | (215996) | (215997) | (215998) | (215999) | (216000) | (216001) |
| 211883 | 78.1 nM | 73.4 nM | 95.7 nM | 55.6 nM | 56.2 nM | 93.1 nM |
| 200053 | 5.5 nM | 8.5 nM | 8.3 nM | n.d. | 6.5 nM | 6.5 nM |
| 77404 | 0.16 nM | 0.4 nM | 0.05 nM | 0.1 nM | 0.3 nM | 0.6 nM |

SEQ ID NO: 2 has 100% identity to 56 amino acids of SEQ ID NO: 1. SPR binding studies with SEQ ID NO: 2 and point mutations in SEQ ID NO: 2 (M2K, or D3F, or F5E, or Y8Q, or A16H, or M23E, or K25R) vs. ubiquitin mutein 77404 showed high affinity binding (below 0.5 nM) to the ubiquitin mutein 77404. SEQ ID NO: 2 is identical to SEQ ID NO: 1 except two N-terminal amino acids that are missing in SEQ ID NO: 2. Thus, the SEQ ID NO: 2_M2K or SEQ ID NO: 2_D3F or SEQ ID NO: 2_F5E or SEQ ID NO: 2_Y8Q or SEQ ID NO: 2_A16H or SEQ ID NO: 2_M23E or SEQ ID NO: 2_K25R have 98% identity to 56 amino acids of SEQ ID NO: 1.

1000 RU 187028 or 1500 RU 187029 proteins were immobilized via C-terminal Cysteine on a CM-5 sensor chip (GE Healthcare) using NHS/EDC after PDEA activation with a Biacore 3000 system (GE Healthcare). The chip was equilibrated with SPR running buffer (PBS 0.05% Tween pH 7.3), Ubiquitin (1 μM; as dimer) was applied to the chip and the affinity was measured as single point. The KD of 187028 vs. ubiquitin was 5.15e−7 M, the KD of 187029 vs. ubiquitin was 2.32e−7 M.

### Example 4. Affinity Purification of Ubiquitin Mutein

Coupling parameter. Purified affinity ligands were immobilized at 30 mg per mL activated Praesto Epoxy 85 (Purolite) according to the manufacturer's instructions, coupling conditions: 35° C. for 3 h, pH 9.5, 110 mg Na₂SO₄ per mL resin. Affinity ligands were successfully coupled to epoxy-activated Praesto 85 resin; the coupling efficiency was 70-76%.

DBC10% and SBC with purified ubiquitin mutein 77404 (SEQ ID NO: 14). DBC Determination was performed with purified immobilized fusion proteins on Praesto 85 epoxy activated (referred to as Praesto_214410; Praesto_214411; or Praesto_214412). Purified ubiquitin mutein 77404 was diluted in PBS at 0.3 mg/ml and adjusted to pH 7.3 and applied onto column with 6 min residence time. Loading of 77404 was followed by column washing with PBS and elution at pH 3.5 in 100 mM citric acid and strip at pH 2.0 in 100 mM citric acid. Eluted ubiquitin mutein 77404 was quantified by UV 280 nm. 100% elution of 77404 was measured at pH 3.5; DBC 10% with 6 min residence time: ˜8 mg/mL, SBC˜6 mg/mL for all three resins.

Determination of pH elution value. 1 mg of ubiquitin mutein 77404 was loaded at 6 min residence time in PBS (pH 7.3). Elution was performed using 100 mM citric acid buffer from pH 6.0 to pH 2.0 in 15 CV. Elution was calculated by UV 280 nm absorption. The maximum peak elution was at pH 3.7 for Praesto_214410, Praesto_214411, and Praesto_214412.

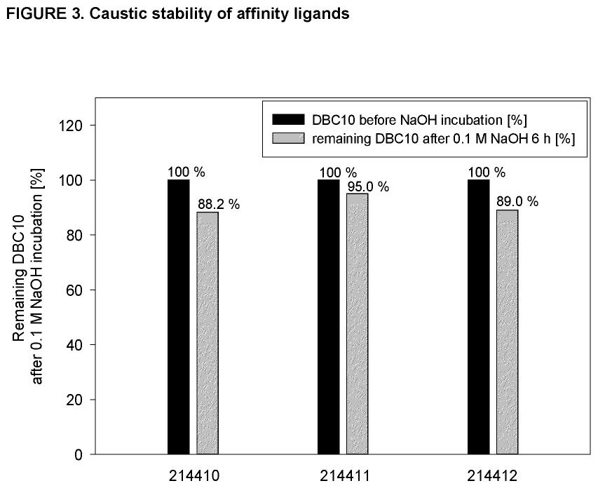

Caustic stability. 214410, 214411, and 214412 were coupled to Praesto™ Epoxy 85, respectively, as described above and treated with 0.1 M NaOH for 6 h at RT. The remaining dynamic binding capacity was determined with ubiquitin mutein 77404 from initial DBC/SBC measurements. The remaining dynamic binding capacity after 6 h 0.1 M NaOH was at least 88%, see FIG. 3.

The point mutation affinity ligands SEQ ID NO: 2 (M2K or D3F or F5E or Y8Q or A16H or M23E or K25R) have 98% identity to 56 amino acids of SEQ ID NO: 1 and are stable under alkaline conditions of 0.25 M NaOH incubation for at least 6 h.

Endotoxin level determination. After coupling 187029 to Praesto™ Epoxy85 as described above the reduction of the endotoxin after one purification step was determined. Therefore E. coli BL21 lysate containing ubiquitin mutein 77404 was purified using the described resin. The endotoxin level of the lysate and after this purification step was measured using the Endosafe® nexgen-PTS™ device and the corresponding cartridges according to the manufacturer manual. Also a comparison with a two-step purification (Streptactin/SEC) was demonstrated.

### Example 5. Purification of Different Ubiquitin Muteins From Affinity Chromatography (AC) Runs—Analytics

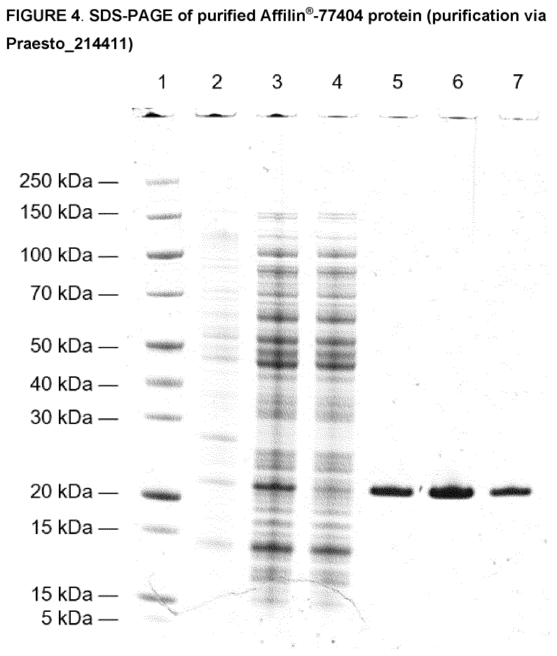

Different ubiquitin muteins were purified using affinity chromatography (214411 coupled to Praesto™ Epoxy 85; Praesto_214411) and eluted by a pH gradient. Eluted fraction of performed AC runs were pooled and pH adjusted using 1 M Tris pH 10.0 to pH 7.4. Purified ubiquitin muteins were analyzed via SDS-PAGE (see FIG. 4). The purity of the ubiquitin mutein purified via a one-step-procedure with Praesto_214411 is comparable between the two-step-process via Strepatactin-AIC and SEC but much more efficient. Analytical results are shown in Table 3.

TABLE 3
Analytical results for affinity chromatography (AC)
purification of ubiquitin muteins vs. Praesto_214411

| | | | | | | | purity |
|---|---|---|---|---|---|---|---|
| SEQ | | | column | | yield | SBC | (SDS- |
| ID | | ubiquitin | volume | elution | (AC) | [mg/ | PAGE) |
| NO: | CID | mutein | (mL) | pH | [mg] | mL] | [%] |
| 10 | 211883 | monomer | 0.39 | 4.5 | 1.18 | 3.0 | 92.6 |
| 11 | 139819 | monomer* | 0.39 | 4.5 | 1.69 | 4.3 | 92.8 |
| 12 | 215208 | monomer* | 0.40 | 3.5 | 0.51 | 1.3 | 83.4 |
| 13 | 215220 | monomer* | 0.41 | 4.3 | 2.20 | 5.4 | 100 |
| 14 | 77404 | dimer | 0.41 | 3.8 | 2.23 | 5.5 | 100 |
| 17 | 200053 | dimer | 0.40 | 4.5 | 1.55 | 3.8 | 100 |

(*refers to a monomer having an additional insertion)

Further, the endotoxin level of the ubiquitin mutein purified in a one-step-process via a resin comprising SEQ ID NO: 1 is 1 log reduction value (LRV) much lower than the endotoxin level of the ubiquitin mutein purified in a conventional two-step-process (Streptactin/SEC) purification.

TABLE 4
Endotoxin levels of ubiquitin mutein (Affilin)
purified via different procedures

| | EU/ml | EU/mg | LRV |
|---|---|---|---|
| BL21 lysate | 2154646 | n.d. | — |
| Affilin purified via | 187 | 175 | 4 |
| conventional two step process | | | |
| Affilin purified via | 6.7 | 10 | 5 |
| one step process via SEQ ID | | | |
| NO: 1 resin | | | |

1. An affinity ligand comprising an amino acid sequence with at least 90% sequence identity to at least 56 amino acids of SEQ ID NO: 1, wherein the affinity ligand is stable under alkaline conditions of 0.1 M NaOH incubation for at least 6 h, and wherein the affinity ligand has a binding affinity of less than 100 nM for a ubiquitin mutein.

2. The affinity ligand according to claim 1, wherein the affinity ligand binds to a ubiquitin mutein having at least 80% identity to ubiquitin of SEQ ID NO: 20.

3. The affinity ligand according to claim 1, wherein the ubiquitin mutein is a monomer or a dimer.

4. The affinity ligand according to claim 4, wherein the ubiquitin mutein has an amino acid sequence with at least 80% identity to any of SEQ ID NOs: 10-19.

5. The affinity ligand according to claim 1, wherein the affinity ligand is fused to at least one non-Immunoglobulin-binding protein.

6. The affinity ligand according to claim 5, wherein the affinity ligand is fused to at least one non-Immunoglobulin-binding protein with an amino acid sequence that is at least 90% identical to SEQ ID NO: 9.

7. (canceled).

8. An affinity separation matrix comprising the affinity ligand according to claim 1.

9. (canceled).

10. A method for producing a ubiquitin mutein comprising at least one chromatographic step employing an affinity chromatography matrix having an affinity for a ubiquitin mutein wherein the affinity ligand according to claim 1 is coupled to said affinity chromatography matrix.

11. A method for affinity purifying a ubiquitin mutein, the method comprising:
(a) providing a liquid that contains a ubiquitin mutein, optionally a ubiquitin mutein with at least 80% amino acid sequence identity to SEQ ID NO: 20;
(b) providing an affinity separation matrix comprising at least one affinity ligand according to claim 1 coupled to said affinity separation matrix;
(c) contacting said affinity separation matrix with the liquid under conditions that permit binding of the at least one affinity ligand according to claim 1; and
(d) eluting said ubiquitin mutein from said affinity purification matrix.

12. The method according to claim 11, wherein in step (d) the elution pH is 3.5 or higher, optionally wherein the elution pH is between 3.5 and 4.5.

13. A method for analyzing for the presence of a ubiquitin mutein in a liquid sample, the method comprising:
(i) providing a liquid that contains a ubiquitin mutein,
(ii) providing an affinity ligand according to claim 1,
(iii) contacting the liquid of (i) with the affinity ligand according to claim 1 under conditions that permit binding of the affinity ligand to the ubiquitin mutein to form a complex comprising the affinity ligand according to claim 1 and the ubiquitin mutein, and
(iv) determining the amount of the complex formed, thereby analyzing for the presence of the ubiquitin mutein in the liquid of (i).

14. A polynucleotide encoding the affinity ligand according to claim 1.
